(19) United States
(12) Patent Application Publication
(10) Pub. No.: US 2024/0150423 A1
(43) Pub. Date: May 9, 2024

(54) GLP-1 ANALOGUE-MODIFIED DIMERS WITH DIFFERENT CONFIGURATIONS, PREPARATION METHOD THEREOF, AND APPLICATION THEREOF IN TREATMENT OF TYPE II DIABETES

(71) Applicant: Nanjing Finepeptide Biopharmaceutical Co.,Ltd, Nanjing (CN)

(72) Inventors: Song-Shan Tang, Nanjing (CN); Qun Luo, Nanjing (CN); Xu-Dong Zhang, Guangzhou (CN); Jing-Xuan TANG, Ann Arbor, MI (US); Li Yang, Guangzhou (CN); Hong-Mei Tan, Guangzhou (CN)

(73) Assignee: Nanjing Finepeptide Biopharmaceutical Co.,Ltd, Nanjing (CN)

(21) Appl. No.: 17/768,236

(22) PCT Filed: Nov. 9, 2020
(86) PCT No.: PCT/CN2020/127422

§ 371 Date: Apr. 12, 2022

(30) Foreign Application Priority Data
Oct. 12, 2019 (CN) 201910969964.X
Nov. 20, 2019 (CN) 201911142332.2

(51) Int. Cl. C07K 14/605; A61P 1/18; A61P 3/10

(52) U.S. Cl. CPC C07K 14/605; A61P 1/18; A61P 3/10; A61K 38/00

(57) ABSTRACT

Applications of different configurations of novel glucagon peptide 1 with fatty acid modification or nonmodification in the treatment of T2D or the pancreas protection are provided. The dimer of the present disclosure is formed by two identical cysteine-containing GLP-1 monomers through a disulfide bond. The H-like GLP-1 homodimer (disulfide bond is inside chains) showed remarkable increase in hypoglycemic duration without reducing specific activity. The GLP-1 dimer provided has an in-vivo effective duration of up to 19 days, which significantly prolonged compared with that of the positive control drug Liraglutide with 3 days of effective duration, or thereby greatly promoting the technical advancement in long-acting GLP-1 drugs and facilitating their clinical applications and business. Meanwhile the U-like homodimer (disulfide bond is at the C-terminus) does not affect blood glucose, but can obviously protect pancreatic exocrine cells such as acini and ducts, and improve pancreas function.

### CROSS REFERENCE TO THE RELATED APPLICATIONS

This application is the national phase entry of International Application No. PCT/CN2020/127422, filed on Nov. 9, 2020, which is based upon and claims priority to Chinese Patent Application No. 201910969964.X, filed on Oct. 12, 2019, and Chinese Patent Application No. 201911142332.2, filed on Nov. 20, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBKY076_Sequence_Listing.txt, created on Apr. 11, 2022, and is 44,098 bytes in size.

### TECHNICAL FIELD

The present disclosure belongs to the field of biopharmaceutics, particularly the preparations and application of a variety of human novel GLP-1 analogue monomers or homodimers in treatment of diabetes.

### BACKGROUND

Glucagon-like peptide 1 (GLP-1) derived from glucagon proprotein is an incretin analogue having 30 amino acid residues, and is released by intestine L cells during the intake of nutrients. It enhances insulin secretion from pancreas β cells, increases expression of insulin and utilization of peripheral glucose, inhibits apoptosis of β cells, promotes satiety and regeneration of β cells, reduces secretion of glucagon and delays gastric emptying. Through these multiple effects, GLP-1 receptor agonists have significance in treatment of type II diabetes. So far, GLP-1 analogues approved by FDA include Liraglutide administered once a day, Exenatide administered twice a day and Albiglutide, Dulaglutide, Exenatide LAR, Lixisenatide, Semaglutide and Taspoglutide administered once a week.

Exendin-4 is an incretin analogue isolated from the saliva of Heloderma suspectum, with 39 amino acids and 53% sequence homology with GLP-1. Exenatide, a synthetic Exendin-4 molecule, has a long half-life (3.3-4.0 h) in anti-hyperglycemic effect and is administered twice a day.

Liraglutide is a GLP-1 analogue, and has 97% homology with natural human GLP-1. It contains Arg→³⁴Lys substitution and a glutamylpalmitoyl chain at ²⁶Lys. After subcutaneous injection, it has an average elimination half-life of 13 h, and allows to be administered once a day, and its pharmacokinetic characteristics are not affected by ages, genders, kidney or liver functions.

PB-105 is produced by replacing cysteine at position 39 of Exenatide and is modified at the cysteine through specific PEGylation to prepare PB-110 (PEG5kd), PB-106 (PEG20kd), PB-107 (PEG30kd) and PB-108 (PEG40kd). PB-106 has a plasma T1/2 that is about 10 times higher than that of PB-105 and therefore exhibits better hypoglycemic activity, but the hypoglycemic activity per milligram (specific activity) is reduced above 90%.

Lixisenatide, a novel long-acting GLP-1R agonist which contains 44 amino acids, is similar to Exendin-4 in structure, except that there is no proline at position 38, and 6 lysine residues are added at position 39. In clinic trial for 24 weeks, Lixisenatide significantly reduced glucagon activity level after being administered once a day, the incidence of side reaction in the Lixisenatide group was similar to that in control group (Lixisenatide 2.5% vs placebo 1.9%), and the symptomatic hypoglycemia rate was 3.4% for Lixisenatide and 1.2% for placebo.

BPI-3016 is produced by structurally modifying the linkage (DIM) between position 8 (Ala) and positions 8-9 (Glu) of human GLP-1, wherein the —CH3 side chain in ⁸Ala is replaced with —CF3, the carbonyl in the linkage is converted into methyl, a palmitoylated Lys→²⁶Arg substitution is used and a C-terminal Gly is added. BPI-3016 showed a half-life of over 95 hours in Diabetic cynomolgus monkeys after a single administration. Obviously after medication for one week, FPG, postprandial blood glucose (PPG), body mass index (BMI), and body fat were reduced, meanwhile the glucose tolerance was improved and the insulin-increasing effect was presented.

Albiglutide, which is a recombinant fusion protein, expressed from two linked copies of the human GLP-1 gene in tandem with the human albumin gene. It is endowed resistance to DPP-4 hydrolysis by using Gly→⁸Ala substitution, and allows to be administered once a week.

Research showed that Albiglutide can reduce blood glucose parameters (HbA1c, PPG, and FPG) by enhancing glucose-dependent insulin secretion and alleviating gastric emptying.

Dulaglutide, which is a GLP-1 analogue fused to a Fc fragment, has a structure of Gly⁸Glu²²Gly³⁶-GLP-1(7-37)-(Gly⁴Ser)₃-Ala-Ala^{234,235}Pro²²⁸-IgG4-Fc. It is administered once a week. Compared with placebo, metformin, insulin glargine, sitagliptin, and Exenatide, Dulaglutide showed a higher HbAlc reduction. Dulaglutide showed various curative effects in the treatment of T2D such as reducing body weight, alleviating nephropathy progression, decreasing the incidence of myocardial infarction, and reducing blood pressure.

Semaglutide, which is a long-acting GLP-1 analogue, has an Aib→⁸Ala substitution and a longer ²⁶Lys linker (2xAEEAC-δ-glutamyl-α-oleic diacid). It maintains 94% homology with GLP-1. Compared with Liraglutide, Semaglutide showed 3 times of hypoglycemic activity reduction and an increasing binding ability with albumin. It was speculated that Semaglutide has a half-life of 165-184 hours (7 days). Semaglutide showed a significant reduction in HbAlc and body weight.

Taspoglutide contains α-aminoisobutyric acid (α-Aib)→⁸Ala and ³⁵Gly with hGLP-1(7-36)NH₂. Taspoglutide has a strong affinity with GLP-1R and a complete resistance to aminodipeptidase. In a clinic trial for 24 weeks, Taspoglutide showed a significant reduction of HbA1c, FPG, and body weight with obvious side effects.

There is still a need for the optimization of GLP-1 analogues, because it has been proven that the current long-acting activators are less effective than Liraglutide or natural GLP-1 in specific activity (hypoglycemic effect per milligram), administration dosage, body weight reduction, and side effects. For example, in a trial for 26 weeks, the body weight was reduced by 0.6 kg for Albiglutide group, by 2.2 kg for Liraglutide group, by 2.9 kg for Dulaglutide group, and by 3.6 kg for Liraglutide group. In rodents, Semaglutide can cause thyroid C-cell tumor on a dose-dependent and time-dependent manners. Clinical trial indicated that renal function was normal in 57.2%, mildly impaired in 35.9%, and moderately impaired in 6.9% of patients. Among patients receiving Semaglutide, gastrointestinal adverse reactions such as nausea, vomiting, diarrhea, abdominal pain, and constipation occurred more frequently than placebo (15.3% for placebo group, 32.7% and 36.4% for Semaglutide 0.5 mg and 1 mg groups). When Semaglutide was used in combination with sulfonylurea, severe hypoglycemia occurred in 0.8-1.2% of patients, injection-site discomfort and erythema occurred in 0.2% of patients. The patients had a mean increase in amylase of 13% and lipase of 22%. Cholelithiasis was occurred in 1.5% and 0.4%, respectively.

### SUMMARY

The present disclosure intends to provide a glucagon-like peptide 1 analogue monomer and homodimers thereof to overcome the above defects in the prior art.

The first purpose of the present disclosure is to provide a glucagon-like peptide 1 analogue monomer having an amino acid sequence that is any one of the following four sequences:
(1) His-X₈-Glu-Gly-Thr-Phe-Thr-Cys-Asp-Val-Ser-Ser-Tyr-Leu-Glu-Gly-Gln-Ala-Ala-X₂₆-Glu-Phe-Ile-Ala-Trp-Leu-Val-X₃₄-X₃₅-Arg-X₃₇ (SEQ ID NO: 1); or
(2) His-X₈-Glu-Gly-Thr-Phe-Thr-Ser-Asp-Val-Cys-Ser-Tyr-Leu-Glu-Gly-Gln-Ala-Ala-X₂₆-Glu-Phe-Ile-Ala-Trp-Leu-Val-X₃₄-X₃₅-Arg-X₃₇ (SEQ ID NO: 2); or
(3) His-X₈-Glu-Gly-Thr-Phe-Thr-Ser-Asp-Val-Ser-Cys-Tyr-Leu-Glu-Gly-Gln-Ala-Ala-X₂₆-Glu-Phe-Ile-Ala-Trp-Leu-Val-X₃₄-X₃₅-Arg-X₃₇ (SEQ ID NO: 3); or
(4) His-X₈-Glu-Gly-Thr-Phe-Thr-Ser-Asp-Val-Ser-Ser-Tyr-Leu-Glu-Gly-Gln-Ala-Ala-X₂₆-Glu-Phe-Ile-Ala-Trp-Leu-Val-X₃₄-X₃₅-Arg-Gly-Cys-OH (SEQ ID NO: 4);
   wherein, X₈ is L-α-alanine (Ala), β-alanine (β-Ala), or α-/β-amino isobutyric acid (α- or β-Aib);
   X₂₆ is lysine, lysine modified with alkanoylglutamyl on the side chain ε-amino, or lysine modified with an alkanoyl on the side chain ε-amino;
   X₃₄ is Arg, Lys, or lysine modified with alkanoylglutamyl on the side chain ε-amino;
   X₃₅ is Gly, Ala, β-alanine, α-amino isobutyric acid or β-amino isobutyric acid;
   X₃₇ is a moiety of Gly-COOH (C-terminal glycine with carboxyl group), Gly-NH₂ (C-terminal glycine with amidation, NH₂ (³⁶arginine with amidation), or OH (³⁶arginine with carboxyl terminus); or the allosteric amino acid sequence of the first 7-36 positions as provided in the first purpose which formed in a copy of one similar repeat sequence, wherein the ⁸alanine (X₈) in the repeat sequence is replaced with glycine or β-/β-amino isobutyric acid (Aib), the cysteine is replaced with serine or glycine, and the X₂₆ in the repeat sequence is arginine; or a PEG-modification by combining the C-terminal amido with a polyethylene glycol molecule, wherein the molecular weight of PEG is 0.5-30 KD.

Preferably, when the X₂₆ is lysine modified with alkanoylglutamyl [γ-Glu(N-α-alkanoyl)] on the side chain ε-amino, it has the structural formula in Formula 1; when the X₂₆ is lysine modified with an alkanoyl on the side chain ε-amino, it has the structural formula in Formula 2, in Formulas 1 and 2, n is equal to 14 or 16:

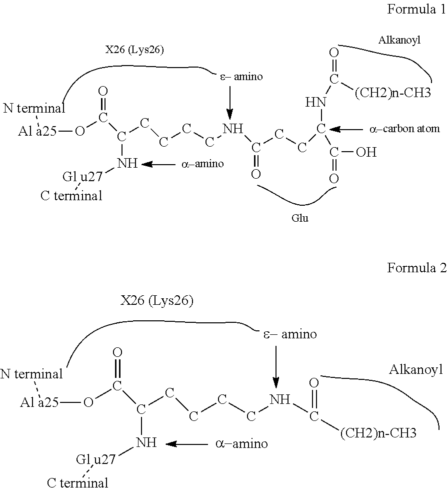

The second purpose of the present disclosure is to provide a glucagon-like peptide 1 analogue homodimer. The dimers are formed by two identical monomers as described above connection through a disulfide bond between cysteines, constitute an H or U-like glucagon-like peptide 1 analogues.

Preferably, the amino acid sequence of the dimer is any one of the following four sequences:

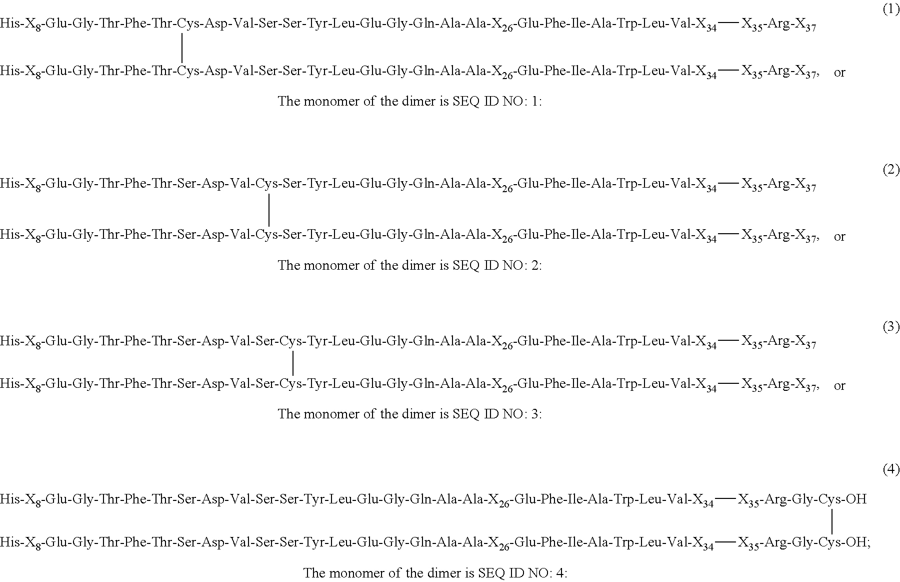

wherein, X₈ is L-α-alanine (Ala), β-alanine (β-ALa), or α-/β-amino isobutyric acid (α- or β-Aib);
X₂₆ is lysine, lysine modified with alkanoylglutamyl on the side chain ε-amino, or lysine modified with an alkanoyl on the side chain ε-amino;
X₃₄ is Arg, Lys, or lysine modified with alkanoylglutamyl on the side chain ε-amino;
X₃₅ is Gly, Ala, β-alanine, or α-/β-amino isobutyric acid (Aib);
X₃₇ is a moiety of Gly-COOH (C-terminal glycine with carboxyl group), Gly-NH₂ (C-terminal glycine with amidation), NH₂ (³⁶arginine with amidation), or OH (³⁶arginine with carboxyl terminus, or an allosteric amino acid sequence of the first 7-36 positions as provided in the first purpose which formed in a copy of one similar repeat sequence, wherein the ⁸alanine (X₈) in the repeat sequence is replaced with a glycine, or α-/β-amino isobutyric acid (Aib), the cysteine is replaced with serine or glycine, and the X₂₆ in the repeat sequence is arginine or a PEG-modification by combining the C-terminal amido with a polyethylene glycol molecule, wherein the molecular weight of the PEG is 0.5-30 KD.

Preferably, when the X₂₆ is lysine modified with alkanoylglutamyl [γ-Glu(N-α-alkanoyl)] on the side chain ε-amino, it has the structural formula in Formula 1; when the X₂₆ is lysine modified with an alkanoyl on the side chain ε-amino, it has the structural formula in Formula 2. In Formulas 1 and 2, n is equal to 14 or 16.

The third purpose of the present disclosure is to provide application of the above monomeric GLP-1 analogue or above dimeric GLP-1 analogue in preparation and of the pancreas protection or/and hypoglycemic drug for treating type II diabetes.

The fourth purpose of the present disclosure is to provide a drug for protecting pancreas or treating type II diabetes, which uses the above monomeric GLP-1 analogue or dimeric GLP-1 analogue as an active content.

The present disclosure has the beneficial effects that the H-like GLP-1 analogue homodimer significantly increases 2-4 times of hypoglycemic time than its single chain GLP 1 analogue i.e. the dimeric peptide had obviously increased specific activity and significantly prolonged the effect of the single chain of GLP-1R agonists approved by FDA. The in-vivo activity duration of the provided GLP-1 analogue homodimer is up to 19 days, which is obviously longer than that of the positive drug Liraglutide. Obviously a technological upgrade is achieved and it will be more benefit in clinical application and market sale. The U-like dimer does not affect blood glucose level, but can obviously protect pancreatic exocrine cells such as pancreas acini and ducts to protect pancreas functions, and thus can be used in treatment of pancreas-related diseases.

### BRIEF DESCRIPTION OF THE DRAWINGS

FIG. 1 shows the results of blood glucose in a single oral glucose tolerance test (single OGTT).

FIG. 2A shows the body weight change of 2G2-2G6 in the multiple OGTT tests.

FIG. 2B shows the body weight change of 2G7-2G8 in the multiple OGTT tests.

FIG. 3 shows the body weight change in T2D model treated with 2G3.

FIG. 4 shows the hypoglycemic effect in T2D model treated with 2G3.

FIG. 5 shows the H-E staining results of the pancreatic tissues of T2D models.

FIG. 6 shows the Ki67 protein expression in T2D model treated with dimeric 2G3.

FIG. 7 shows the Ki67 protein expression in T2D model treated with dimeric 2G1.

FIG. 8 shows the TUNEL staining results.

FIG. 9 shows the GLP-1R staining results.

FIG. 10 shows the Western blot results of GLP-1R.

FIG. 11A shows the insulin staining.

FIG. 11B shows the insulin staining analysis.

FIG. 11C shows the pancreatic islet number analysis.

### DETAILED DESCRIPTION OF THE EMBODIMENTS

To more clearly illustrate the technical solution, objectives, and advantages of the present disclosure, the present disclosure will be described in detail in combination with specific examples and figures below.

### Embodiment 1 Preparations of Monomeric and Dimeric Peptides

### I. Solid-Phase Synthesis Process of Monomeric Peptide: Manual Solid-Phase Peptide Synthesis Process.

1. Resin swelling: a 2-Chorotrityl Chloridie resin (final product with C-terminal carboxyl) or an aminomethyl resin (final product with C-terminal amidation) (purchased from Tianjin Nankai Hecheng Science & Technology Co., Ltd, China) was put into a reaction pot. Dichloromethane (DCM, Dikma Technologies Inc., China) was added in the pot in the volume 15 ml/g resin. The pot was oscillated for 30 min. A SYMPHONY® 12-channel polypeptide synthesizer (SYMPHONY®, software Version.201, Protein Technologies Inc., China) was used in the synthsis.
2. Addition of the first amino acid: the solvent was removed by suction filtration with a sand core funnel. The first Fmoc-AA amino acid at the C-terminus (all Fmoc-amino acids were provided by Suzhou Tianma Pharmaceutical Group Fine Chemicals Co., Ltd., China) was added in 3 times of mole, subsequently 10 times of mole of 4-dimethylaminopyridine (DMAP) and N,N′-dicyclohexylcarbodiimide (DCC) were added in the pot. Finally, dimethylformamide (DMF) (purchased from dikma Technologies Inc.) was added to dissolve and oscillate for 30 min. The block was used in acetic anhydride.
3. Deprotection: In order to remove DMF, a 20% piperidine-DMF solution (15 ml/g) was added and oscillated for 5 min, the solvent was removed by filtration, and once more 20% piperidine-DMF solution (15 ml/g) was added and oscillated for 15 min. Piperidine was provided by Shanghai Chemical Reagent Company of Sinopharm group in China.
4. Detection: the solvent was removed by suction. A dozen of resin particles were taken and washed three times with ethanol, added with one drop of ninhydrin, KCN, and phenol solutions respectively, and heated for 5 min at 105-110° C. Dark blue indicated positive.
5. Resin washing: In turns the resin was washed twice with DMF (10 ml/g), twice with methanol (10 ml/g), and twice with DMF (10 ml/g) in turn.
6. Condensation: According to specific synthesis conditions, the following methods can be used alone or in combination during synthesis of a polypeptide:
Method a: 3 times of Fmoc amino acid and 2-(7-azobenzotriazole)-tetramethylurea hexafluorophosphate (HBTU, Suzhou Tianma Pharmaceutical Group Fine Chemicals Co., Ltd in China) were dissolved with as little DMF as possible, and then added into the reaction pot. 10 times of N-methylmorpholine (NMM, Suzhou Tianma Pharmaceutical Group Fine Chemical Co., Ltd.) was instantly added to react for 30 min. Detection showed negative.

Method b: 3 times of FMOC-AA and 1-hydroxybenzotriazole (HOBt, Suzhou Tianma Pharmaceutical Group Fine Chemicals Co., Ltd.) were dissolved with as little DMF as possible, and then added into a reaction tube. N,N′-diisopropylcarbodiimide (DIC) in 3 times was instantly added to react for 30 min. Detection showed negative.
7. Resin washing: The resin was washed once with DMF (10 ml/g), twice with methanol (10 ml/g), and twice with DMF (10 ml/g) in turn.

8. The operations in steps 2-6 were repeated to successively combine correct amino acids as GLP-1 peptide sequence without or with side chain modification shown in Table 1 from the right to the left. The peptides with K₂₆ or K₃₄ modification were synthesized according to the method in 9 below.
9. Synthesis of K₂₆ or K₃₄ [N-ε-(N-α-alkanoyl-L-γ-glutamyl)]: 10 ml of 2% hydrazine hydrate was added to react for 30 min to remove the protecting group Dde of Fmoc-Lys(Dde)-OH and expose the amino in the side chain. The resin was washed with DMF and methanol alternately for six times, the ninhydrin detection showed blue. 550 mg of Fmoc-GLU-OTBU and 250 mg of HOBT were mixed and dissolved with DMF, subsequently 0.3 ml of DIC was added. The mixture solution was added into the reactor to react with the side chain amino of lysine for 1 h. After suction filtered to dryness, the resin was washed four times with DMF, and the ninhydrin detection showed negative. 5 ml of 20% piperidine solution in DMF was added into the reactor to react for 20 min to remove the N-terminal protection group Fmoc of Fmoc-GLU-OTBU. The resin was washed with DMF and methanol alternately for six times, and the ninhydrin detection showed positive blue. 300 mg of palmitic acid and 250 mg of HOBT were mixed and dissolved with DMF, subsequently 0.3 ml of DIC was added into the reactor for 1 h. After suction filtered to dryness, the resin was washed four times with DMF, and the ninhydrin detection showed negative colorless. The resin was washed twice with methanol and then suction filtered to dryness.
Synthesis of K₂₆ and/or K₃₄ [Nε-(N-α-alkanoyl)]: When K[N-ε-(alkanoyl)] was needed to be synthesized, the series of reaction steps above for adding Fmoc-γ-Glu(tbu)-OH were omitted. After the fmoc group in Dde-Lys(fmoc) was removed, an alkanoyl was connected directly. Reaction was conducted for 30 min with 2% hydrazine hydrate to remove the Dde group of Dde-Lys and K₂₆ and/or K₃₄ modifying residues were connected via the above step 8.
10. The polypeptide was washed twice with DMF (10 ml/g), twice with DCM (10 ml/g) and twice with DMF (10 ml/g) in turn, and then suction filtered to dryness for 10 min. The Ninhydrin detection showed negative.
11. The FMOC group of the N-terminal amino acid of the peptide chain was removed. Detection showed positive, and the solution was filtered sucking to dryness.
12. The resin was washed according to the following protocol. twice with DMF (10 ml/g), twice with methanol (10 ml/g), twice DMF (10 ml/g), and twice with DCM (10 ml/g) in turn, and then suction filtered to dryness for 10 min.
13. Cutting polypeptides from the resin: a cutting solution (10 ml/g) was prepared: TFA 94.5% (J. T. Baker Chemical Company); water 2.5%, ethanedithiol (EDT, Sigma-Aldrich Chemistry) 2.5% and triisopropylsilane (TIS, Sigma-Aldrich Chemistry) 1%. Cutting time: 120 min.
14. For PEG modified monomeric peptide, when nonmodified monomeric peptide was synthesized according to the above methods and the polypeptide was cutted as C-terminal amide, a Fmoc-PAL-PEG-PS was reacted with the monomeric peptide for chemical solid-phase synthesis. After the synthesis was finished, the obtained polypeptide resin with the side chain PEG group was cleaved to obtain a PEG-modified monomeric peptide, the PEG had a molecular weight of 0.5-30 KD.
15. Blow-dry and wash: The lysate was blow-dried as possible with nitrogen, washed six times with diethylether, and then evaporated to dryness at room temperature.
16. The polypeptide was purified and identified in high performance liquid chromatography (HPLC) below, and then stored in the dark at −20° C.

### II. Preparation of Monomeric Peptides in a Combinatorial Method of Genetic Recombination-Chemical Modification:

Some monomeric peptides claimed herein can be synthesized in the above solid-phase synthesis, or also done through the genetic recombination combining with chemical modification as the examples of G3 and G9 syntheses below.
Genetic Recombination: After the DNA sequence of the allosteric G3 or G9 monomeric peptide or its one or two copies was inserted into a pMD-18 plasmid, the recombinant plasmide was digested in KPN I and ExoRI and larger segment was purified. The bank pET32a plasmid was also doubly digested and then the larger segments was harvested. The gene segments of the target peptide was linked to the larger segment of pET32a by T4 ligase to obtain a recombinant expression vector pET32a/Trx-EK-G3. The recombinant vector was transformed in host bacterial BL21 using the CaCl₂ method. The TRX-EK-G3 monomer peptide fusion protein was expressed by inducing in 0.5 mM IPTG. After being purified via Ni-Sepharose chromatography, The TRX-EK (thioredoxin-enterokinase) was then removed by enterokinase digestion. The recombinant monomeric peptide was purified with a C18 reverse-phase column and lyophilized.
Side Chain Lysine Chemical Modification: 0.01 mmol of the monomeric peptide lyophilized powder (only with a single ²⁶Lys structure) was dissolved into 5 ml of water at 4° C., and adjusted pH to 12.5 with a sodium hydroxide solution. After 2 min, 5 ml of NMP and 20 μl of triethylamine were added in turn. The solution was adjusted pH 10.5 in 1M acetic acid solution at 15° C. The N-palmitoyl-(or oleoyl)-L-glutamic acid-5-succinimide-1-methyl ester (0.012 mmol) was added in the solution for 2.5 h. The mixture was adjusted pH to 12.8 with sodium hydroxide solution and then hydrolyzed at 15° C. to remove methoxy group for 2 h. The mixture was adjusted pH to 6.8 in 1 M acetic acid solution. The mixture was put on a C4 column and the column was eluted to remove NMP in 5% acetonitrile-water solution, and then eluted in 50% acetonitrile-water solution. The eluent was concentrated in vacuum-rotary evaporation and subsequently purified with RP-HPLC to obtain a purity of 95% or above. After the sample was lyophilized, a palmitoylated or oleoylated GLP-1 analogue monomer was harvested.

The identification method is following:
1. Purification of polypeptide in HPLC: The crude peptide was dissolved in pure water or in a little amount of acetonitrile, and then purified based on the following conditions: HPLC apparatus (analytical type; software Class VP, Sevial System, Shimadzu Co, Japan) and Venusi MRC-ODS C18 chromatographic column (30×250 mm, Tianjin Bonna-Agela Technologies). Mobile phase A: 0.1% trifluoroacetic acid aqueous solution and mobile phase B: 0.1% trifluoroacetic acid-99.9% acetonitrile solution (acetonitrile was purchased from Fisher Scientific Company). Flow rate: 1 ml/min, loading volume: 30 μL, and detection wavelength: 220 nm. Elution procedure: 0-5 min: 90% phase A+10% phase B; 5-30 min: 90% phase A/10% phase B→20% phase A/80% phase B.
2. Finally, the effective fraction was lyophilized in a lyophilizer (lyophilizer Freezone Plus 6, manufacturer LABCONCO) to obtain the finished product.
3. Identification: a small amount of the final polypeptide was put on HPLC to analyze its purity: Chromatographic column (4.6×150 mm). Mobile phase A: 0.1% trifluoroacetic acid aqueous solution and mobile phase B: 99.9% acetonitrile-0.1% trifluoroacetic acid solution, flow rate: 1 ml/min, loading volume: 10 μl, detection wavelength: 220 nm. Elution procedure: 0-5 min: 100% phase A; 5-30 min: 100% phase A→20% phase A/80% phase B. The required purity determinated is 95% or above. The specific method refers to our authorized patent (Chinese patent ZL201410612382.3).
Mass Spectrum (MS) Identification of molecular weight of the polypeptide: A polypeptide with a qualified purity was dissolved in water, and added in 5% acetic acid, 8% acetonitrile, 87% water in turn, The sample was subjected to electrospray ionization mass spectrometry (EI-MS) for the molecular weight. The specific method refers to our authorized patent (China Patent ZL201410612382.3).
4. The polypeptide powder was sealed and stored in the dark at −20° C.
Formation of dimer: 1 mg/ml of final monomeric polypeptide with only one cysteine at the C-terminus was dissolved in an aqueous solution of pH 9.5 and kept for 4 h at 37° C. to form a homodimeric peptide. The dimeric peptide was isolated in G-25 chromatographic column (2×60 cm, the dimeric fraction was the first peak). The dimeric peptide can be identified by peptide PAGE electrophoresis with thiol-free reagent or EI-MS. The specific method refers to our authorized patent (Chinese patent ZL201410612382.3).

Some GLP-1 analogue monomers and the dimers were synthesized in our laboratory and some peptides were done by commercial companies. The inventor confirmed the peptide structure by using HPLC purity, EIMS, and cysteine oxidation. The amino acid sequences of the GLP-1 analogue monomers and the homodime were showed in the present disclosure as shown in Tables 1 and 2.

### Embodiment 2 Durability of Hypoglycemic Effects of GLP-1 Monomers (G2-9 Series) and Homodimers (2G2-9 series) of the Present Disclosure

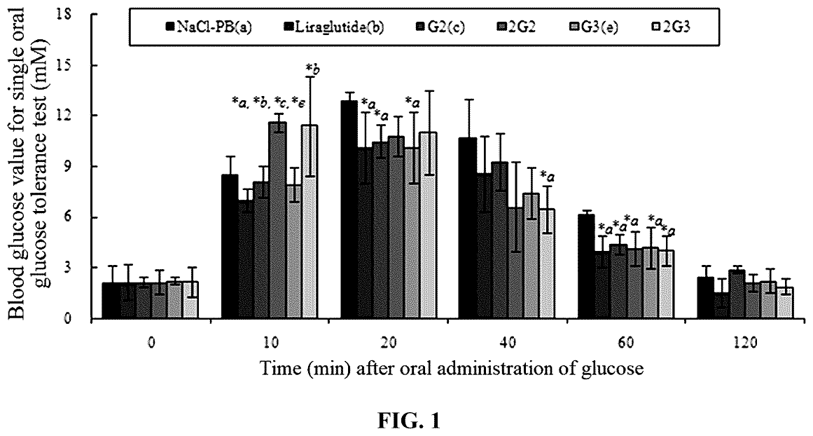

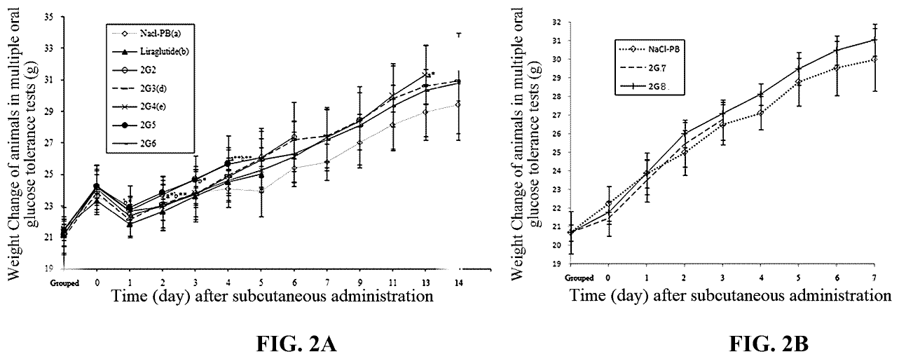

1. Methods: Normal Kunming (KM) mice (purchased from Guangdong Animal Center) were used for oral glucose tolerance test (OGTT) to obtain the hypoglycemic activity and durability of drug. According to the result of non-different fasting blood glucose, male KM mice (5 weeks old) were grouped (NaCl-PB group, Liraglutide group, monomer G2-G9 series groups, and dimer 2G2-2G9 series groups) (n=6). After an adaptation period including two rounds of 14-hour feeding-10-hour fasting, the KM mice immediately underwent an OGTT after the second 10-hour fasting. 30 min after being subcutaneously injected with the same molar dose of monomeric or dimeric peptide at the back, the mice were orally administered with a 5% glucose solution in gastric lavage, and the blood glucose value of the mouse tail was measured accurately at 35 min. The blood glucose meter and blood glucose test paper were from Bayer HeathCare LLC. The average blood glucose of each group was used as an evaluation standard: When the average blood glucose of experimental group was higher than that of the NaCl control group for two times, the measurement stopped and the hypoglycemic period compared to the blank control group was the effective duration.
2. Results
2.1 single OGTTs: after a single administration, the mice were orally administered glucose once and measured the blood glucose from the tail blood at 0, 10, 20, 40, 60 and 120 min. The results of the single OGTT (sOGTT) showed that a glucose peak appeared within 10 min for the 2G2 or 2G3 group, while no blood glucose peak appeared in NaCl-PB, Liraglutide, G2, and G3 groups, indicating that the dimers significantly delay the absorption. With more time, the hypoglycemic effect of 2G2 or 2G3 was stronger than that of monomeric G2 or G3, but there is no significant difference between them (FIG. 1).
2.2 multiple OGTT: After a single administration of the same molar dose (1.126 nmol), multiple OGTT (mOGTT) was continued for several days. The hypoglycemic duration results of the monomers G2-9 and dimers 2G2-9 are shown in Tables 1 and 2. Based on the average blood glucose as evaluation standard, the effective duration was 3 days for the positive drug Liraglutide, 3-13 days for the 2G2 series groups, 14-17 days for the 2G3 series, 12-18 days for the 2G4, only 3-8 days for the 2G5 series, 16-19 days for the 2G6, 2-7 days for the 2G7 series, 2-8 days for the 2G8 series, and 4-5 days for the 2G9 series. The duration of each monomer group was about ½-¼ of the duration of its corresponding dimer group. In this test, G9 and 2G9 serial groups had significant hypoglycemic activities and short durations at the same dose due to the C-terminal extension compared with the NaCl-PB and Liraglutide group. The mice in the 2G4, 2G5, 2G7, and 2G8 serial groups had significantly high weights (P<0.05 or 0.01, 0.001) (FIGS. 2A-2B). By comparison, it was found that the dimeric peptides of 2G3 and 2G6 serial groups had a longer duration up to 19 days. The 2G3 peptide in the 2G3 series not only showed hypoglycemic activity lasting for 14 days, but also had the most obvious weight loss. In addition, Liraglutide, with which the 2G3 has the highest sequence identity, was selected as the positive control drug. Therefore, the 2G3 peptide was selected for treating type II diabetes (T2D) in vivo and subsequent experiments.

TABLE 1
Amino acid sequences of the novel GLP-1 monomeric peptides synthesized in the
present disclosure and their hypoglycemic duration for a single
injection at the same dose (1.126 nmol)

| kind of | SEQ | | Hypoglycemic |
|---|---|---|---|
| peptide | ID NO: | Sequence of monomeric peptide | (day) |
| Lira- | 5 | His-Ala-Glu-Gly-Thr-Phe-Thr-Ser-Asp-Val-Ser-Ser-Tyr-Leu-Glu- | 3 |
| glutide | | Gly-Gln-Ala-Ala-Lys[N-ϵ-(N-α-Palmitoyl-γ-glutamyl)]-Glu- | |
| | | Phe-Ile-Ala-Trp-Leu-Val-Arg-Gly-Arg-Gly-OH | |
| G1 | 6 | His-Ala-Glu-Gly-Thr-Phe-Thr-Ser-Asp-Val-Ser-Ser-Tyr-Leu-Glu- | No hypoglycemic |
| | | Gly-Gln-Ala-Ala-Lys[N-ϵ-(N-α-Palmitoyl-γ-glutamyl)]-Glu-Phe- | activity |
| | | Ile-Ala-Trp-Leu-Val-Arg-Gly-Arg-Gly-Cys-OH | (G1 peptide) |
| G2 | 7 | His-Ala-Glu-Gly-Thr-Phe-Thr-Ser-Asp-Val-Cys-Ser-Tyr-Leu-Glu- | |
| series | | Gly-Gln-Ala-Ala-Lys[N-ϵ-(N-α-Palmitoyl-L-γ-glutamyl)]-Glu- | |
| | | Phe-Ile-Ala-Trp-Leu-Val-Arg-Gly-Arg-Gly-OH | |
| | 8 | His-Ala-Glu-Gly-Thr-Phe-Thr-Ser-Asp-Val-Cys-Ser-Tyr-Leu-Glu- | 5 |
| | | Gly-Gln-Ala-Ala-Lys[N-ϵ-(N-α-Palmitoyl-L-γ-glutamyl)]-Glu- | |
| | | Phe-Ile-Ala-Trp-Leu-Val-Arg-Gly-Arg-Gly-NH₂ | |
| | 9 | His-Ala-Glu-Gly-Thr-Phe-Thr-Ser-Asp-Val-Cys-Ser-Tyr-Leu-Glu- | 3 |
| | | Gly-Gln-Ala-Ala-Lys[N-ϵ-(N-α-oleoyl-L-γ-glutamyl)]-Glu-Phe- | |
| | | Ile-Ala-Trp-Leu-Val-Arg-Gly-Arg-Gly-OH | |
| | 10 | His-Ala-Glu-Gly-Thr-Phe-Thr-Ser-Asp-Val-Cys-Ser-Tyr-Leu-Glu- | 5 |
| | | Gly-Gln-Ala-Ala-Lys[N-ϵ-(N-α-oleoyl-L-γ-glutamyl)]-Glu-Phe- | |
| | | Ile-Ala-Trp-Leu-Val-Arg-Gly-Arg-Gly-NH₂ | |
| | 11 | His-aAib-Glu-Gly-Thr-Phe-Thr-Ser-Asp-Val-Cys-Ser-Tyr-Leu-Glu- | 4 |
| | | Gly-Gln-Ala-Ala-Lys[N-ϵ-(N-α-Palmitoyl-γ-glutamyl)]-Glu-Phe- | |
| | | Ile-Ala-Trp-Leu-Val-Arg-Gly-Arg-Gly-OH | |
| | 12 | His-aAib-Glu-Gly-Thr-Phe-Thr-Ser-Asp-Val-Cys-Ser-Tyr-Leu-Glu- | 6 |
| | | Gly-Gln-Ala-Ala-Lys[N-ϵ-(N-α-Palmitoyl-γ-glutamyl)]-Glu-Phe- | |
| | | Ile-Ala-Trp-Leu-Val-Arg-Gly-Arg-Gly-NH₂ | |
| | 13 | His-aAib-Glu-Gly-Thr-Phe-Thr-Ser-Asp-Val-Cys-Ser-Tyr-Leu-Glu- | 7 |
| | | Gly-Gln-Ala-Ala-Lys[N-ϵ-(N-α-oleoyl-L-γ-glutamyl)]-Glu-Phe- | |
| | | Ile-Ala-Trp-Leu-Val-Arg-Gly-Arg-Gly-NH₂ | |
| | 14 | His-aAib-Glu-Gly-Thr-Phe-Thr-Ser-Asp-Val-Cys-Ser-Tyr-Leu-Glu- | 4 |
| | | Gly-Gln-Ala-Ala-Lys[N-ϵ-(N-α-oleoyl-L-γ-glutamyl)]-Glu-Phe- | |
| | | Ile-Ala-Trp-Leu-Val-Arg-Gly-Arg-Gly-OH | |
| | 15 | His-βAib-Glu-Gly-Thr-Phe-Thr-Ser-Asp-Val-Cys-Ser-Tyr-Leu-Glu- | 5 |
| | | Gly-Gln-Ala-Ala-Lys[N-ϵ-(N-α-oleoyl-L-γ-glutamyl)]-Glu-Phe- | |
| | | Ile-Ala-Trp-Leu-Val-Arg-Gly-Arg-Gly-NH₂ | |
| | 16 | His-βAib-Glu-Gly-Thr-Phe-Thr-Ser-Asp-Val-Cys-Ser-Tyr-Leu-Glu- | 3 |
| | | Gly-Gln-Ala-Ala-Lys-Glu-Phe-Ile-Ala-Trp-Leu-Val-Arg-βAib-Arg-NH₂ | |
| | 17 | His-βAib-Glu-Gly-Thr-Phe-Thr-Ser-Asp-Val-Cys-Ser-Tyr-Leu-Glu- | 3 |
| | | Gly-Gln-Ala-Ala-Lys-Glu-Phe-Ile-Ala-Trp-Leu-Val-Lys-βAib-Arg-NH₂ | |
| | 18 | His-βAla-Glu-Gly-Thr-Phe-Thr-Ser-Asp-Val-Cys-Ser-Tyr-Leu-Glu- | 5 |
| | | Gly-Gln-Ala-Ala-Lys[N-ϵ-(N-α-oleoyl-L-γ-glutamyl)]-Glu-Phe-Ile- | |
| | | Ala-Trp-Leu-Val-Arg-Gly-Arg-Gly-NH₂ | |
| | 19 | His-βAla-Glu-Gly-Thr-Phe-Thr-Ser-Asp-Val-Cys-Ser-Tyr-Leu-Glu-G | 3 |
| | | ly-Gln-Ala-Ala-Lys-Glu-Phe-Ile-Ala-Trp-Leu-Val-Arg-βAla-Arg-NH₂ | |
| G3 | 20 | His-Ala-Glu-Gly-Thr-Phe-Thr-Ser-Asp-Val-Ser-Cys-Tyr-Leu-Glu-Gly- | 4 (G3 peptide) |
| series | | Gln-Ala-Ala-Lys[N-ϵ-(N-α-Palmitoyl-L-γ-glutamyl)]-Glu-Phe-Ile- | |
| | | Ala-Trp-Leu-Val-Arg-Gly-Arg-Gly-OH | |
| | 21 | His-Ala-Glu-Gly-Thr-Phe-Thr-Ser-Asp-Val-Ser-Cys-Tyr-Leu-Glu-Gly- | 4 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-OH | |
| | 22 | His-Ala-Glu-Gly-Thr-Phe-Thr-Ser-Asp-Val-Ser-Cys-Tyr-Leu-Glu-Gly- | 6 |
| | | Gln-Ala-Ala-Lys[N-ϵ-(N-α-Palmitoyl-L-γ-glutamyl)]-Glu-Phe-Ile- | |
| | | Ala-Trp-Leu-Val-Arg-Gly-Arg-Gly-NH₂ | |
| | 23 | His-Ala-Glu-Gly-Thr-Phe-Thr-Ser-Asp-Val-Ser-Cys--Tyr-Leu-Glu-Gly- | 7 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-NH₂ | |
| G4 | 24 | His-Ala-Glu-Gly-Thr-Phe-Thr-Ser-Asp-Val-Cys-Ser-Tyr-Leu-Glu-Gly- | 3 |
| series | | Gln-Ala-Ala-Lys[N-ϵ-(N-α-Palmitoyl)]-Glu-Phe-Ile-Ala-Trp-Leu-Val- | |
| | | Arg-Gly-Arg-Gly-OH | |
| | 25 | His-Ala-Glu-Gly-Thr-Phe-Thr-Ser-Asp-Val-Cys-Ser-Tyr-Leu-Glu-Gly- | 5 |
| | | Gln-Ala-Ala-Lys[N-ϵ-(N-α-Palmitoyl)]-Glu-Phe-Ile-Ala-Trp-Leu-Val- | |
| | | Arg-Gly-Arg-Gly-NH₂ | |
| | 26 | His-Ala-Glu-Gly-Thr-Phe-Thr-Ser-Asp-Val-Cys-Ser-Tyr-Leu-Glu-Gly- | 4 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-OH | |
| | 27 | His-Ala-Glu-Gly-Thr-Phe-Thr-Ser-Asp-Val-Cys-Ser-Tyr-Leu-Glu-Gly- | 6 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 28 | His-aAib-Glu-Gly-Thr-Phe-Thr-Ser-Asp-Val-Cys-Ser-Tyr-Leu-Glu-Gly- | 7 |
| | | Gln-Ala-Ala-Lys[N-ϵ-(N-α-Palmitoyl)]-Glu-Phe-Ile-Ala-Trp-Leu-Val- | |
| | | Arg-Gly-Arg-Gly-NH₂ | |
| | 29 | His-aAib-Glu-Gly-Thr-Phe-Thr-Ser-Asp-Val-Cys-Ser-Tyr-Leu-Glu-Gly- | 7 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 30 | His-βAib-Glu-Gly-Thr-Phe-Thr-Ser-Asp-Val-Cys-Ser-Tyr-Leu-Glu-Gly- | 6 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 31 | His-βAla-Glu-Gly-Thr-Phe-Thr-Ser-Asp-Val-Cys-Ser-Tyr-Leu-Glu-Gly- | 5 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 32 | His-Ala-Glu-Gly-Thr-Phe-Thr-Ser-Asp-Val-Ser-Cys-Tyr-Leu-Glu-Gly- | 5 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-OH | |
| | 33 | His-Ala-Glu-Gly-Thr-Phe-Thr-Ser-Asp-Val-Ser-Cys-Tyr-Leu-Glu-Gly- | 7 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 34 | His-Ala-Glu-Gly-Thr-Phe-Thr-Ser-Asp-Val-Ser-Cys-Tyr-Leu-Glu-Gly- | 4 |
| | | Gln-Ala-Ala-Lys[N-ϵ-(N-α-Palmitoyl)]-Glu-Phe-Ile-Ala-Trp-Leu-Val- | |
| | | Arg-Gly-Arg-Gly-OH | |
| | 35 | His-Ala-Glu-Gly-Thr-Phe-Thr-Ser-Asp-Val-Ser-Cys-Tyr-Leu-Glu-Gly- | 6 |
| | | Gln-Ala-Ala-Lys[N-ϵ-(N-α-Palmitoyl)]-Glu-Phe-Ile-Ala-Trp-Leu-Val- | |
| | | Arg-Gly-Arg-Gly-NH₂ | |
| | 36 | His-aAib-Glu-Gly-Thr-Phe-Thr-Ser-Asp-Val-Ser-Cys-Tyr-Leu-Glu-Gly- | 8 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 37 | His-aAib-Glu-Gly-Thr-Phe-Thr-Ser-Asp-Val-Ser-Cys-Tyr-Leu-Glu-Gly- | 7 |
| | | Gln-Ala-Ala-Lys[N-ϵ-(N-α-Palmitoyl)]-Glu-Phe-Ile-Ala-Trp-Leu-Val- | |
| | | Arg-Gly-Arg-Gly-NH₂ | |
| | 38 | His-βAib-Glu-Gly-Thr-Phe-Thr-Ser-Asp-Val-Ser-Cys-Tyr-Leu-Glu-Gly- | 6 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| | 39 | His-βAla-Glu-Gly-Thr-Phe-Thr-Ser-Asp-Val-Ser-Cys-Tyr-Leu-Glu-Gly- | 5 |
| | | Gln-Ala-Ala-Lys[N-ϵ-(N-α-oleoyl)]-Glu-Phe-Ile-Ala-Trp-Leu-Val-Arg- | |
| | | Gly-Arg-Gly-NH₂ | |
| G5 | 40 | His-Ala-Glu-Gly-Thr-Phe-Thr-Ser-Asp-Val-Ser-Cys-Tyr-Leu-Glu-Gly- | 1 |
| series | | Gln-Ala-Ala-Lys[N-ϵ-(N-α-Palmitoyl-L-γ-glutamyl)]-Glu-Phe-Ile-Ala- | |
| | | Trp-Leu-Val-Lys[N-ϵ-(N-α-Palmitoyl-L-γ-glutamyl)]-Gly-Arg-Gly-OH | |
| | 41 | His-Ala-Glu-Gly-Thr-Phe-Thr-Ser-Asp-Val-Ser-Cys-Tyr-Leu-Glu-Gly- | 3 |
| | | Gln-Ala-Ala-Lys[N-ϵ-(N-α-Palmitoyl-L-γ-glutamyl)]-Glu-Phe-Ile-Ala- | |
| | | Trp-Leu-Val-Lys[N-ϵ-(N-α-Palmitoyl-L-γ-glutamyl)]-Gly-Arg-Gly-NH₂ | |
| | 42 | His-aAib-Glu-Gly-Thr-Phe-Thr-Ser-Asp-Val-Ser-Cys-Tyr-Leu-Glu-Gly- | 3 |
| | | Gln-Ala-Ala-Lys[N-ϵ-(N-α-Palmitoyl-L-γ-glutamyl)]-Glu-Phe-Ile-Ala- | |
| | | Trp-Leu-Val-Lys[N-ϵ-(N-α-Palmitoyl-L-γ-glutamyl)]-Gly-Arg-Gly-NH₂ | |
| | 43 | His-βAib-Glu-Gly-Thr-Phe-Thr-Ser-Asp-Val-Ser-Cys-Tyr-Leu-Glu-Gly- | 3 |
| | | Gln-Ala-Ala-Lys[N-ϵ-(N-α-Palmitoyl-L-γ-glutamyl)]-Glu-Phe-Ile-Ala- | |
| | | Trp-Leu-Val-Lys[N-ϵ-(N-α-Palmitoyl-L-γ-glutamyl)]-Gly-Arg-Gly-NH₂ | |
| | 44 | His-βAla-Glu-Gly-Thr-Phe-Thr-Ser-Asp-Val-Ser-Cys-Tyr-Leu-Glu-Gly- | 2 |
| | | Gln-Ala-Ala-Lys[N-ϵ-(N-α-Palmitoyl-L-γ-glutamyl)]-Glu-Phe-Ile-Ala- | |
| | | Trp-Leu-Val-Lys[N-ϵ-(N-α-Palmitoyl-L-γ-glutamyl)]-Gly-Arg-Gly-NH₂ | |
| G6 | 45 | His-aAib-Glu-Gly-Thr-Phe-Thr-Ser-Asp-Val-Ser-Cys-Tyr-Leu-Glu-Gly- | 4 |
| series | | Gln-Ala-Ala-Lys[N-ϵ-(N-α-Palmitoyl-L-γ-glutamyl)]-Glu-Phe-Ile-Ala- | |
| | | Trp-Leu-Val-Arg-Gly-Arg-Gly-OH | |
| | 46 | His-aAib-Glu-Gly-Thr-Phe-Thr-Ser-Asp-Val-Ser-Cys-Tyr-Leu-Glu-Gly- | 5 |
| | | Gln-Ala-Ala-Lys[N-ϵ-(N-α-oleoyl-L-γ-glutamyl)]-Glu-Phe-Ile-Ala-Trp- | |
| | | Leu-Val-Arg-Gly-Arg-Gly-OH | |
| | 47 | His-aAib-Glu-Gly-Thr-Phe-Thr-Ser-Asp-Val-Ser-Cys--Tyr-Leu-Glu-Gly- | 8 |
| | | Gln-Ala-Ala-Lys[N-ϵ-(N-α-oleoyl-L-γ-glutamyl)]-Glu-Phe-Ile-Ala-Trp- | |
| | | Leu-Val-Arg-Gly-Arg-Gly-NH₂ | |
| | 48 | His-aAib-Glu-Gly-Thr-Phe-Thr-Ser-Asp-Val-Ser-Cys-Tyr-Leu-Glu-Gly- | 7 |
| | | Gln-Ala-Ala-Lys[N-ϵ-(N-α-Palmitoyl-L-γ-glutamyl)]-Glu-Phe-Ile-Ala- | |
| | | Trp-Leu-Val-Arg-Gly-Arg-Gly-NH₂ | |
| | 49 | His-βAib-Glu-Gly-Thr-Phe-Thr-Ser-Asp-Val-Ser-Cys-Tyr-Leu-Glu-Gly- | 5 |
| | | Gln-Ala-Ala-Lys[N-ϵ-(N-α-oleoyl-L-γ-glutamyl)]-Glu-Phe-Ile-Ala-Trp- | |
| | | Leu-Val-Arg-Gly-Arg-Gly-NH₂ | |
| | 50 | His-βAla-Glu-Gly-Thr-Phe-Thr-Ser-Asp-Val-Ser-Cys-Tyr-Leu-Glu-Gly- | 4 |
| | | Gln-Ala-Ala-Lys[N-ϵ-(N-α-oleoyl-L-γ-glutamy)]-Glu-Phe-Ile-Ala-Trp- | |
| | | Leu-Val-Arg-Gly-Arg-Gly-NH₂ | |
| G7 | 51 | His-aAib-Glu-Gly-Thr-Phe-Thr-Ser-Asp-Val-Ser-Cys-Tyr-Leu-Glu-Gly- | 3 |
| series | | Gln-Ala-Ala-Lys-Glu-Phe-Ile-Ala-Trp-Leu-Val-Arg-aAib-Arg-NH₂ | |
| | 52 | His-aAib-Glu-Gly-Thr-Phe-Thr-Ser-Asp-Val-Ser-Cys-Tyr-Leu-Glu-Gly- | 1 |
| | | Gln-Ala-Ala-Lys-Glu-Phe-Ile-Ala-Trp-Leu-Val-Arg-aAib-Arg-OH | |
| | 53 | His-aAib-Glu-Gly-Thr-Phe-Thr-Ser-Asp-Val-Ser-Cys-Tyr-Leu-Glu-Gly- | 3 |
| | | Gln-Ala-Ala-Lys-Glu-Phe-Ile-Ala-Trp-Leu-Val-Lys-aAib-Arg-NH₂ | |
| | 54 | His-βAib-Glu-Gly-Thr-Phe-Thr-Ser-Asp-Val-Ser-Cys-Tyr-Leu-Glu-Gly- | 2 |
| | | Gln-Ala-Ala-Lys-Glu-Phe-Ile-Ala-Trp-Leu-Val-Arg-βAib-Arg-NH₂ | |
| | 55 | His-βAla-Glu-Gly-Thr-Phe-Thr-Ser-Asp-Val-Ser-Cys-Tyr-Leu-Glu-Gly- | 2 |
| | | Gln-Ala-Ala-Lys-Glu-Phe-Ile-Ala-Trp-Leu-Val-Arg-βAla-Arg-NH₂ | |
| G8 | 56 | His-Ala-Glu-Gly-Thr-Phe-Thr-Cys-Asp-Val-Ser-Ser-Tyr-Leu-Glu-Gly- | 1 |
| series | | Gln-Ala-Ala-Lys[N-ϵ-(N-α-Palmitoyl-L-γ-glutamyl)]-Glu-Phe-Ile- | |
| | | Ala-Trp-Leu-Val-Arg-Gly-Arg-Gly-OH | |
| | 57 | His-Ala-Glu-Gly-Thr-Phe-Thr-Cys-Asp-Val-Ser-Ser-Tyr-Leu-Glu-Gly- | 2 |
| | | Gln-Ala-Ala-Lys[N-ϵ-(N-α-Palmitoyl-L-γ-glutamyl)]-Glu-Phe-Ile- | |
| | | Ala-Trp-Leu-Val-Arg-Gly-Arg-Gly-NH₂ | |
| | 58 | His-Ala-Glu-Gly-Thr-Phe-ThrCys-Asp-Val-Ser-Ser-Tyr-Leu-Glu-Gly- | 1 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-OH | |
| | 59 | His-Ala-Glu-Gly-Thr-Phe-Thr-Cys-Asp-Val-Ser-Ser-Tyr-Leu-Glu-Gly- | 3 |
| | | Gln-Ala-Ala-Lys[N-α-(N-α-oleoyl-L-γ-glutamyl)]-Glu-Phe-Ile-Ala- | |
| | | Trp-Leu-Val-Arg-Gly-Arg-Gly-NH₂ | |
| | 60 | His-aAib-Glu-Gly-Thr-Phe-Thr-Cys-Asp-Val-Ser-Ser-Tyr-Leu-Glu-Gly- | 2 |
| | | Gln-Ala-Ala-Lys[N-ϵ-(N-α-Palmitoyl-γ-glutamyl)]-Glu-Phe-Ile-Ala- | |
| | | Trp-Leu-Val-Arg-Gly-Arg-Gly-OH | |
| | 61 | His-aAib-Glu-Gly-Thr-Phe-Thr-Cys-Asp-Val-Ser-Ser-Tyr-Leu-Glu-Gly- | 4 |
| | | Gln-Ala-Ala-Lys[N-ϵ-(N-α-Palmitoyl-γ-glutamyl)]-Glu-Phe-Ile-Ala- | |
| | | Trp-Leu-Val-Arg-Gly-Arg-Gly-NH₂ | |
| | 62 | His-aAib-Glu-Gly-Thr-Phe-Thr-Cys-Asp-Val-Ser-Ser-Tyr-Leu-Glu-Gly- | 5 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-NH₂ | |
| | 63 | His-aAib-Glu-Gly-Thr-Phe-Thr-Cys-Asp-Val-Ser-Ser-Tyr-Leu-Glu-Gly- | 2 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-OH | |
| | 64 | His-aAib-Glu-Gly-Thr-Phe-Thr-Cys-Asp-Val-Ser-Ser-Tyr-Leu-Glu-Gly- | 2 |
| | | Gln-Ala-Ala-Lys-Glu-Phe-Ile-Ala-Trp-Leu-Val-Arg-aAib-Arg-NH₂ | |
| | 65 | His-aAib-Glu-Gly-Thr-Phe-Thr-Cys-Asp-Val-Ser-Ser-Tyr-Leu-Glu-Gly- | 2 |
| | | Gln-Ala-Ala-Lys-Glu-Phe-Ile-Ala-Trp-Leu-Val-Lys-aAib-Arg-NH₂ | |
| | 66 | His-βAib-Glu-Gly-Thr-Phe-Thr-Cys-Asp-Val-Ser-Ser-Tyr-Leu-Glu-Gly- | 3 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-NH₂ | |
| | 67 | His-βAib-Glu-Gly-Thr-Phe-Thr-Cys-Asp-Val-Ser-Ser-Tyr-Leu-Glu-Gly- | 2 |
| | | Gln-Ala-Ala-Lys-Glu-Phe-Ile-Ala-Trp-Leu-Val-Arg-βAib-Arg-NH₂ | |
| | 68 | His-βAla-Glu-Gly-Thr-Phe-Thr-Cys-Asp-Val-Ser-Ser-Tyr-Leu-Glu-Gly- | 4 |
| | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-NH₂ | |
| | 69 | His-βAla-Glu-Gly-Thr-Phe-Thr-Cys-Asp-Val-Ser-Ser-Tyr-Leu-Glu-Gly- | 2 |
| | | Gln-Ala-Ala-Lys-Glu-Phe-Ile-Ala-Trp-Leu-Val-Arg-βAla-Arg-NH₂ | |
| G9 | 70 | His-Ala-Glu-Gly-Thr-Phe-Thr-Ser-Asp-Val-Ser-Cys-Tyr-Leu-Glu-Gly- | 5 (G9 peptide) |
| series | | Gln-Ala-Ala-Lys[N-ϵ-(N-α-oleoyl-L-γ-glutamyl)]-Glu-Phe-Ile-Ala- | |
| | | Trp-Leu-Val-Arg-Gly-Arg-Gly-His-Gly-Glu-Gly-Thr-Phe-Thr-Ser-Asp- | |
| | | Val-Ser-Ser-Tyr-Leu-Glu-Gly-Gln-Ala-Ala-Arg-Glu-Phe-Ile-Ala-Trp- | |
| | | Leu-Val-Arg-Gly-Arg-Gly-OH | |
| | 71 | His-Ala-Glu-Gly-Thr-Phe-Thr-Ser-Asp-Val-Ser-Cys-Tyr-Leu-Glu-Gly- | 3 |
| | | Gln-Ala-Ala-Lys-Glu-Phe-Ile-Ala-Trp-Leu-Val-Arg-Gly-Arg-NH-PEG | |

TABLE 2
Sequences of the novel GLP-1 dimers and their hypoglycemic duration for a single
injection at the same dose (1.126 nmol)

| | SEQ | | |
|---|---|---|---|
| | ID | | |
| | NO: | | Hypo- |
| Number | of the | | glycemic |
| of | mono- | | duration |
| peptide | mer | Sequence of dimeric peptide | (days) |
| Liraglut | 5 | His-Ala-Glu-Gly-Thr-Phe-Thr-Ser-Asp-Val-Ser-Ser-Tyr-Leu-Glu-Gly-Gln-Ala-Ala-Lys[N-ε-(N-α-Palmitoyl-γ-glutamyl)]-Glu-Phe-Ile-Ala-Trp-Leu-Val-Arg-Gly-Arg-Gly-OH | 3 |
| de | | | |
| 2G1 | 6 | 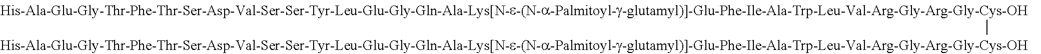 | No hypo- glycemic activity |
| | | | (2G1 |
| | | | peptide) |
| 2G2 series | 7 | 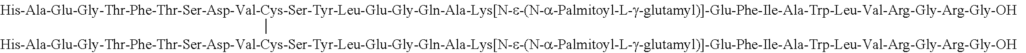 | 5 (2G2 peptide) |
| | 8 | 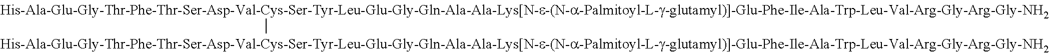 | 7 |
| | 9 | 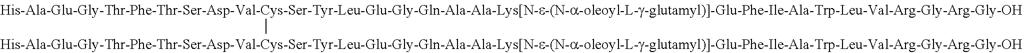 | 5 |
| | 10 | 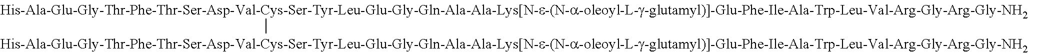 | 8 |
| | 11 | 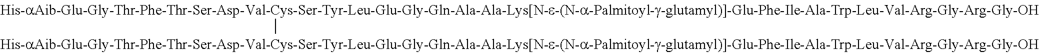 | 9 |
| | 12 | 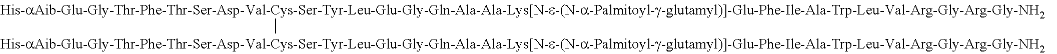 | 11 |
| | 13 | 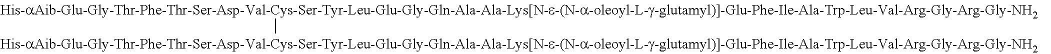 | 13 |
| | 14 | 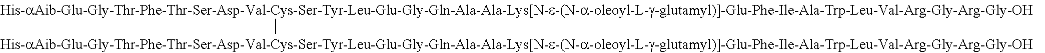 | 9 |
| | 15 | 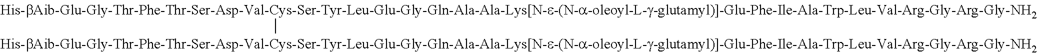 | 10 |
| | 16 | 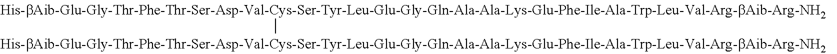 | 4 |
| | 17 | 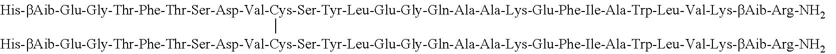 | 4 |
| | 18 | 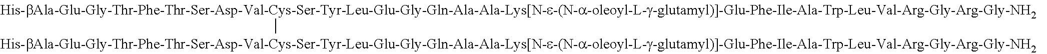 | 9 |
| | 19 | 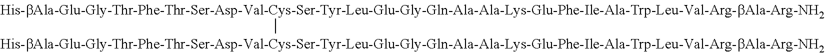 | 3 |
| 2G3 series | 20 | 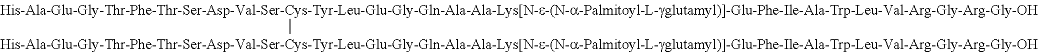 | 14 (2G3 peptide) |
| | 21 | 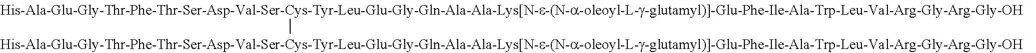 | 14 |
| | 22 | 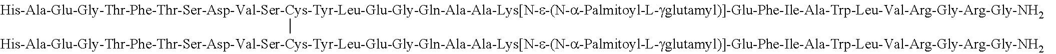 | 16 |
| | 23 | His-Ala-Glu-Gly-Thr-Phe-Thr-Ser-Asp-Val-Ser-Cys--Tyr-Leu-Glu-Gly-Gln-Ala-Ala-Lys[N-ε-(N-α-oleoyl-L-γ-glutamyl)] -Glu-Phe-Ile-Ala-Trp-Leu-Val-Arg-Gly-Arg-Gly-NH₂ | 17 |
| | | His-Ala-Glu-Gly-Thr-Phe-Thr-Ser-Asp-Val-Ser-Cys--Tyr-Leu-Glu-Gly-Gln-Ala-Ala-Lys[N-ε-(N-α-oleoyl-L-γ-glutamyl)] -Glu-Phe-Ile-Ala-Trp-Leu-Val-Arg-Gly-Arg-Gly-NH₂ | |
| 2G4 | 24 | His-Ala-Glu-Gly-Thr-Phe-Thr-Ser-Asp-Val-Cys-Ser-Tyr-Leu-Glu-Gly-Gln-Ala-Ala-Lys[N-ε-(N-α-Palmitoyl)]-Glu-Phe-Ile-Ala-Trp-Leu-Val-Arg-Gly-Arg-Gly-OH | 12 (2G4 |
| series | | His-Ala-Glu-Gly-Thr-Phe-Thr-Ser-Asp-Val-Cys-Ser-Tyr-Leu-Glu-Gly-Gln-Ala-Ala-Lys[N-ε-(N-α-Palmitoyl)]-Glu-Phe-Ile-Ala-Trp-Leu-Val-Arg-Gly-Arg-Gly-OH | peptide) |
| | 25 | His-Ala-Glu-Gly-Thr-Phe-Thr-Ser-Asp-Val-Cys-Ser-Tyr-Leu-Glu-Gly-Gln-Ala-Ala-Lys[N-ε-(N-α-Palmitoyl)]-Glu-Phe-Ile-Ala-Trp-Leu-Val-Arg-Gly-Arg-Gly-NH₂ | 14 |
| | | His-Ala-Glu-Gly-Thr-Phe-Thr-Ser-Asp-Val-Cys-Ser-Tyr-Leu-Glu-Gly-Gln-Ala-Ala-Lys[N-ε-(N-α-Palmitoyl)]-Glu-Phe-Ile-Ala-Trp-Leu-Val-Arg-Gly-Arg-Gly-NH₂ | |
| | 26 | His-Ala-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-OH | 12 |
| | | His-Ala-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-OH | |
| | 27 | His-Ala-Glu-Gly-Thr-Phe-Thr-Ser-Asp-Val-Cys-Ser-Tyr-Leu-Glu-Gly-Gln-Ala-Ala-Lys[N-ε-(N-α-oleoyl)] -Glu-Phe-Ile-Ala-Trp-Leu-Val-Arg-Gly-Arg-Gly-NH₂ | 15 |
| | | His-Ala-Glu-Gly-Thr-Phe-Thr-Ser-Asp-Val-Cys-Ser-Tyr-Leu-Glu-Gly-Gln-Ala-Ala-Lys[N-ε-(N-α-oleoyl)] -Glu-Phe-Ile-Ala-Trp-Leu-Val-Arg-Gly-Arg-Gly-NH₂ | |
| | 29 | His-αAib-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-NH₂ | 18 |
| | | His-αAib-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-NH₂ | |
| | 30 | His-βAib-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-NH₂ | 16 |
| | | His-βAib-Glu-Gly-Thr-Phe-Thr-Ser-Asp-Val-Cys-Ser-Tyr-Leu-Glu-Gly-Gln-Ala-Ala-Lys[N-ε-(N-α-oleoyl)]-Glu-Phe-Ile-Ala-Trp-Leu-Val-Arg-Gly-Arg-Gly-NH₂ | |
| | 31 | 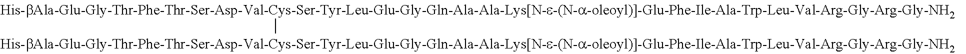 | 15 |
| | 32 | 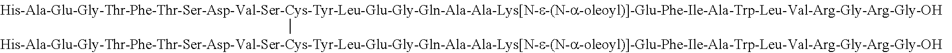 | 13 |
| | 33 | 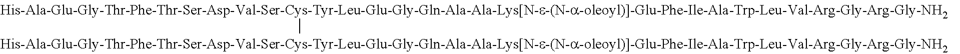 | 16 |
| | 34 | 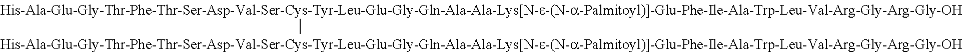 | 13 |
| | 35 | 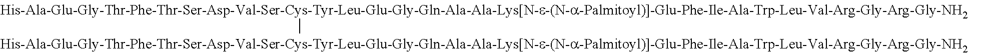 | 15 |
| | 36 | 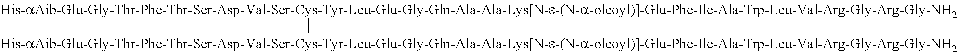 | 18 |
| | 38 | 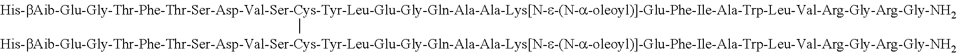 | 17 |
| | 39 | 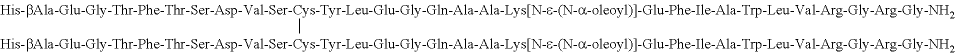 | 16 |
| 2G5 series | 40 | 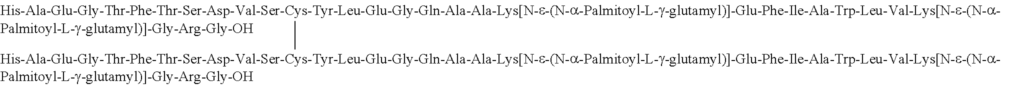 | 3 (2G5 peptide) |
| | 41 | 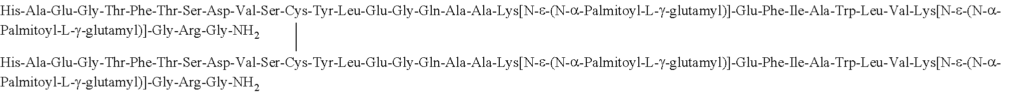 | 5 |
| | 42 | 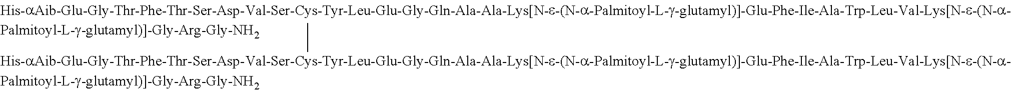 | 8 |
| | 43 | 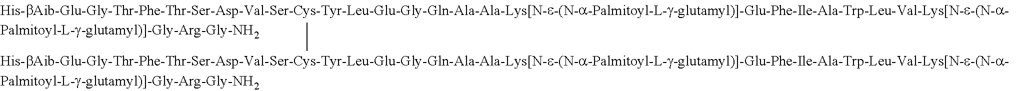 | 7 |
| | 44 | 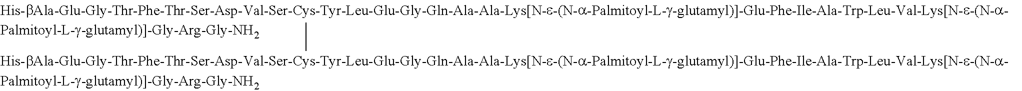 | 6 |
| 2G6 series | 45 | 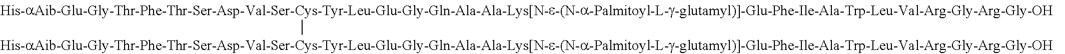 | 17 (2G6 peptide) |
| | 46 | 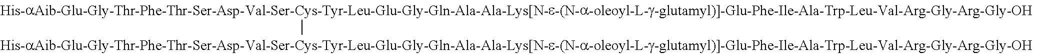 | 18 |
| | 47 | 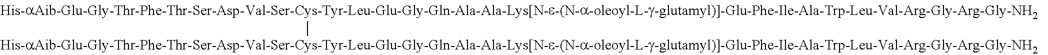 | 19 |
| | 48 | 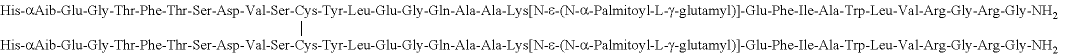 | 18 |
| | 49 | 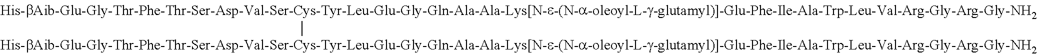 | 17 |
| | 50 | 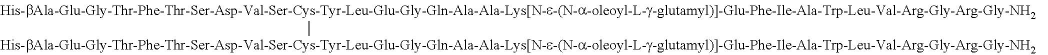 | 16 |
| 2G7 series | 51 | 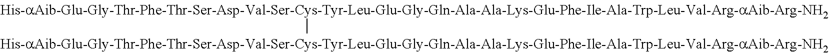 | 7 |
| | 52 | 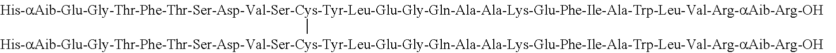 | 2 (2G7 peptide) |
| | 53 | His-αAib-Glu-Gly-Thr-Phe-Thr-Ser-Asp-Val-Ser-Cys-Tyr-Leu-Glu-Gly-Gln-Ala-Ala-Lys-Glu-Phe-Ile-Ala-Trp-Leu-Val-Lys-αAib-Arg-NH₂ | 7 (2G8 |
| | | His-αAib-Glu-Gly-Thr-Phe-Thr-Ser-Asp-Val-Ser-Cys-Tyr-Leu-Glu-Gly-Gln-Ala-Ala-Lys-Glu-Phe-Ile-Ala-Trp-Leu-Val-Lys-αAib-Arg-NH₂ | peptide) |
| | 54 | His-βAib-Glu-Gly-Thr-Phe-Thr-Ser-Asp-Val-Ser-Cys-Tyr-Leu-Glu-Gly-Gln-Ala-Ala-Lys-Glu-Phe-Ile-Ala-Trp-Leu-Val-Arg-βAib-Arg-NH₂ | 5 |
| | | His-βAib-Glu-Gly-Thr-Phe-Thr-Ser-Asp-Val-Ser-Cys-Tyr-Leu-Glu-Gly-Gln-Ala-Ala-Lys-Glu-Phe-Ile-Ala-Trp-Leu-Val-Arg-βAib-Arg-NH₂ | |
| | 55 | His-βAla-Glu-Gly-Thr-Phe-Thr-Ser-Asp-Val -Ser-Cys-Tyr-Leu-Glu-Gly-Gln-Ala-Ala-Lys-Glu-Phe-Ile-Ala-Trp-Leu-Val-Arg-βAla-Arg-NH₂ | 4 |
| | | His-βAla-Glu-Gly-Thr-Phe-Thr-Ser-Asp-Val -Ser-Cys-Tyr-Leu-Glu-Gly-Gln-Ala-Ala-Lys-Glu-Phe-Ile-Ala-Trp-Leu-Val-Arg-βAla-Arg-NH₂ | |
| 2G8 | 56 | His-Ala-Glu-Gly-Thr-Phe-Thr-Cys-Asp-Val-Ser-Ser-Tyr-Leu-Glu-Gly-Gln-Ala-Ala-Lys[N-ε-(N-α-Palmitoyl-L-γ-glutamyl)]-Glu-Phe-Ile-Ala-Trp-Leu-Val-Arg-Gly-Arg-Gly-OH | 2 |
| series | | His-Ala-Glu-Gly-Thr-Phe-Thr-Cys-Asp-Val-Ser-Ser-Tyr-Leu-Glu-Gly-Gln-Ala-Ala-Lys[N-ε-(N-α-Palmitoyl-L-γ-glutamyl)]-Glu-Phe-Ile-Ala-Trp-Leu-Val-Arg-Gly-Arg-Gly-OH | |
| | 57 | His-Ala-Glu-Gly-Thr-Phe-Thr-Cys-Asp-Val-Ser-Ser-Tyr-Leu-Glu-Gly-Gln-Ala-Ala-Lys[N-ε-(N-α-Palmitoyl-L-γ-glutamyl)]-Glu-Phe-Ile-Ala-Trp-Leu-Val-Arg-Gly-Arg-Gly-NH₂ | 5 |
| | | His-Ala-Glu-Gly-Thr-Phe-Thr-Cys-Asp-Val-Ser-Ser-Tyr-Leu-Glu-Gly-Gln-Ala-Ala-Lys[N-ε-(N-α-Palmitoyl-L-γ-glutamyl)]-Glu-Phe-Ile-Ala-Trp-Leu-Val-Arg-Gly-Arg-Gly-NH₂ | |
| | 58 | His-Ala-Glu-Gly-Thr-Phe-Thr-Cys-Asp-Val-Ser-Ser-Tyr-Leu-Glu-Gly-Gln-Ala-Ala-Lys[N-ε-(N-α-oleoyl-L-γ-glutamyl)]-Glu-Phe-Ile-Ala-Trp-Leu-Val-Arg-Gly-Arg-Gly-OH | 3 |
| | | His-Ala-Glu-Gly-Thr-Phe-Thr-Cys-Asp-Val-Ser-Ser-Tyr-Leu-Glu-Gly-Gln-Ala-Ala-Lys[N-ε-(N-α-oleoyl-L-γ-glutamyl)]-Glu-Phe-Ile-Ala-Trp-Leu-Val-Arg-Gly-Arg-Gly-OH | |
| | 59 | His-Ala-Glu-Gly-Thr-Phe-Thr-Cys-Asp-Val-Ser-Ser-Tyr-Leu-Glu-Gly-Gln-Ala-Ala-Lys[N-ε-(N-α-oleoyl-L-γ-glutamyl)]-Glu-Phe-Ile-Ala-Trp-Leu-Val-Arg-Gly-Arg-Gly-NH₂ | 5 |
| | | His-Ala-Glu-Gly-Thr-Phe-Thr-Cys-Asp-Val-Ser-Ser-Tyr-Leu-Glu-Gly-Gln-Ala-Ala-Lys[N-ε-(N-α-oleoyl-L-γ-glutamyl)]-Glu-Phe-Ile-Ala-Trp-Leu-Val-Arg-Gly-Arg-Gly-NH₂ | |
| | 60 | 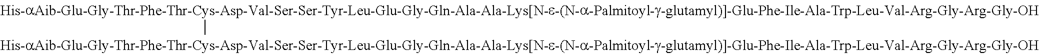 | 4 |
| | 61 | 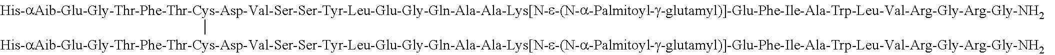 | 6 |
| | 62 | 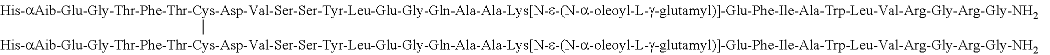 | 8 |
| | 63 | 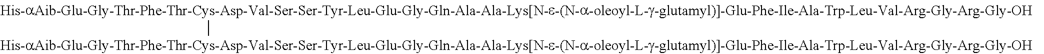 | 5 |
| | 64 | 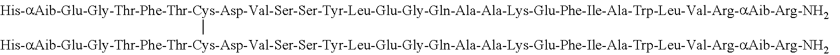 | 3 |
| | 65 | 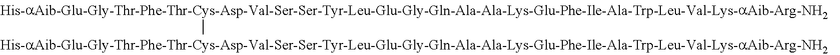 | 3 |
| | 66 | 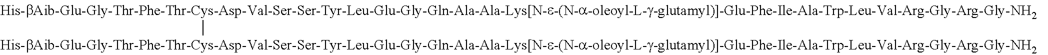 | 8 |
| | 67 | 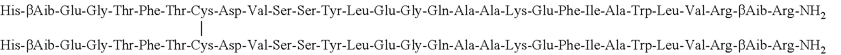 | 3 |
| | 68 | 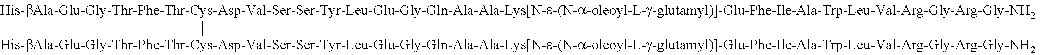 | 7 |
| | 69 | 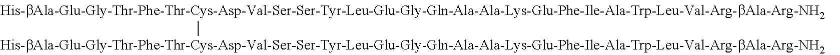 | 3 |
| 2G9 series | 70 | 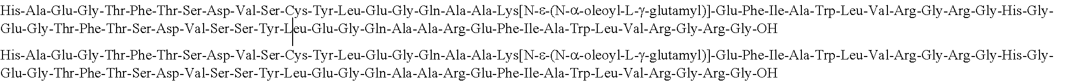 | 11 |
| | 71 | 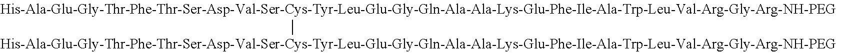 | 5 |

Note:
In the tables, ²⁶Lys[N-ε-(N-α-palmitoyl-L-γ-glutamyl)] and ²⁶Lys[N-ε-(N-α-oleoyl-L-γ-glutamyl)] represent ²⁶lysine modified with alkanoylglutamyl [γ-Glu(N-α-alkanoyl)] on the ε-amino of the side chain;
³⁴Lys[N-ε-(N-α-Palmitoyl)] and ³⁴Lys[N-ε-(N-α-oleoyl)] represent ³⁴lysine modified with an alkanoyl on the ε-amino of the side chain;
Palmitoyl and Oleoyl represent an alkanoyl with 16 or 18 carbons, respectively;
PEG modifies the C-terminal amide group of the monomeric peptide;
″|″ represents a disulfide bond formed between two cysteines in a dimer;
″(GI peptide), (G9 peptide), (G2 peptide), (G3 peptide), (2G1 peptide) and (2G2 peptide), (2G3 peptide), (2G4 peptide), (2G5 peptide), (2G6 peptide), (2G7 peptide), or (2G8 peptide)″ in Tables 1 and 2 was selected as a representative of its corresponding series and completed other experiments. The names in these experiments and figures consistently correspond to them.

### Embodiment 3 Therapeutic Effect of the Dimers on Type II Diabetes Models

### I. Preparation of Type II Diabetes (T2D) Mice Models.

C57B16/J mice were placed in an SPF-level environment with a standard diet. All experimental operations were performed in accordance with the guidelines of the ethics and uses of experimental animals. After feeding several days according to the standard diet, 5-week-old C57B16/J male mice were grouped: NaCl-PB, T2D model control, Liraglutiade, low, medium and high dose dimeric peptide 2G3 or 2G1 groups. The NaCl-PB group served as blank control and the model control group served as the T2D model control. They were injected with NaCl-PB solution. The T2D model group was fed with 60 kcal % high-fat diet (D12492, Changzhou SYSE Bio-tech. Co., Ltd., China) until the end of the experiment. Meanwhile the blank control group maintained standard diet. The preparation method of the diabetic models: After 4 weeks of high-fat feeding, the mice were injected intraperitoneally with 75 mg/kg dose of streptozotocin (STZ, Sigma Chemical Company, USA), and re-injected intraperitoneally with 50 mg/kg dose of STZ after 3 days, mice with blood glucose value equal to or higher than 11 mM after 3 weeks were considered as diabetic mice. These groups were subjected to a 35-day treatment study on the basis of the high-fat diet.

### II. Therapeutic Effect on Type II Diabetes.

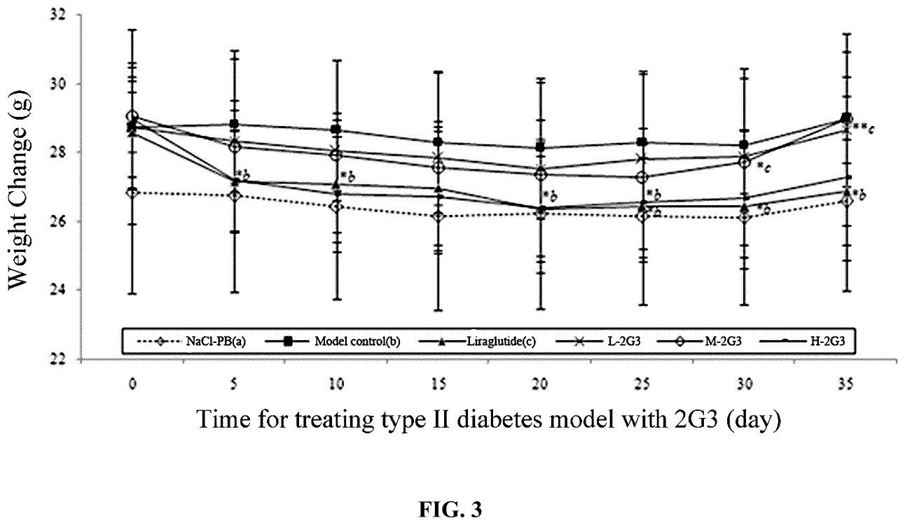

1. Solubility of peptide: The monomeric peptides without Aib showed a suspended state in water, whereas their homodimeric peptides were completely dissolved in water. The monomer peptides with Aib were completely dissolved in water, whereas their homodimeric peptides showed slightly poor solubility in water. The peptides with C-terminal amidation were more insoluble than the peptides with C-terminal COOH structure. All dimeric peptides can achieve high solubility in NaCl-PB (pH 8.0) solution, The different doses (low, medium, and high doses) of 2G3 or 2G1 peptide were dissolved in a saline buffered to pH 8.0 in Na₂HPO₄ (NaCl-PB) for animal injection. The monomeric peptides were dissolved into a saline solution (pH 6.5) for injection.
2. Setting of administration dose: Preliminary experiments revealed that 1.126 nmol of Liraglutide can induce postprandial blood glucose values of 9-11 mM in the T2D diabetes models (blood glucose up to 20 mM). Under this threshold, the effect-dose relationships of the positive drug Liraglutide and the GLP-1 dimers are readily observed. In the OGTT, normal KM mice were injected subcutaneously with a single dose of 1.126 nmol of Liraglutide, or a monomeric peptide, a dimeric peptide at the back hip. The models were measured blood glucose at the tail and weight weighted every morning at 9 am. Because the structure of the 2G3 dimer is similar to that of Liraglutide, the latter as the positive drug and its dosage in clinic was selected. In the T2D treatment study, all T2D model mice were injected subcutaneously at the back hip in 100 μl per mouse within 30 min. The blood glucose of the experimental mice was measured every five days and the whole measurement was completed within 40 min. The high, medium and low doses of the dimeric 2G3 or 2G1 peptide were 3.378, 1.126 and 0.375 nmol/100 μL once in a day, respectively, and the dose of the positive drug Liraglutide was 1.126 nmol/100 μL (4.225 μg/100 μL, stored at −20° C., product batch number: No. 8-9695-03-201-1, Novo Nordisk company, Switzerland) until the end of the 35-day experiment.
3. Weight change after T2D treatment: Before administration, the body weight of the T2D model group was at least 2 g higher than that of the NaCl-PB group and there was no significant difference between the T2D model groups. Compared with the model control group, the body weight of the Liraglutide group decreased rapidly (P<0.05) after the 5^{th}, 20^{th}, 25^{th}, 30^{th}, and 35^{th} day. The body weight of 2G3 peptide groups showed a dose-dependent decrease and the H-2G3 (high dose) group was similar to the Liraglutide group (FIG. 3). 2G1, as a U-like dimer, had no significant effect on the body weight of the model mice, and had significant different in body weight with 2G3 as an H-like dimer.
4. Weight change of organs in the T2D model treatment: In the experiments, Liraglutide led to weight loss, including heart, kidney, liver, and adipose tissues, confirming the mechanism of Liraglutide's stronger regulation in diet. The 2G3 experimental groups showed a dose-dependent decrease and the 2G3 high-dose group was similar to the Liraglutide group in body weight, but the weights of certain organs increased, such as left kidney, right testis, and adipose tissues. 2G3 increased weights of liver and spleen (Table 3). Compared with the Liraglutide group, or/and NaCl-PB group, or T2D model control group, the 2G1 groups had significantly increase in liver, spleen, and adipose tissue weight, or decrease in right testis and pancreas weights (P<0.05, 0.01 or 0.001) (Table 3).

TABLE 3
Comparison of T2D model organ weights (mean ± SD, n ± 10)

| | Weight of | NaCl-PB | Model Control | Liraglutide | Low dose | Medium dose | High dose |
|---|---|---|---|---|---|---|---|
| Dimer | organs (g) | Group | Group | Group | Group | Group | Group |
| 2G3 | Heart | 0.1307 ± 0.03 | 0.1342 ± 0.02 | 0.1152 ± 0.02b* | 0.1199 ± 0.01 | 0.1207 ± 0.01 | 0.1235 ± 0.02 |
| | Liver | 0.962 ± 0.08 | 1.212 ± 0.20a** | 0.940 ± 0.05b*** | 1.138 ± 0.18a*c** | 1.048 ± 0.06a*b*c *** | 1.023 ± 0.12b*c* |
| | Spleen | 0.0678 ± 0.01 | 0.0819 ± 0.01a* | 0.0718 ± 0.01 | 0.0794 ± 0.02 | 0.0807 ± 0.01a* | 0.0763 ± 0.01 |
| | Lung | 0.1449 ± 0.03 | 0.1509 ± 0.01 | 0.1496 ± 0.02 | 0.1565 ± 0.02 | 0.1429 ± 0.02 | 0.1523 ± 0.02 |
| | Left kidney | 0.1525 ± 0.03 | 0.1672 ± 0.02 | 0.1602 ± 0.01 | 0.1703 ± 0.02 | 0.1750 ± 0.02c* | 0.1661 ± 0.02 |
| | Right kidney | 0.1472 ± 0.03 | 0.1695 ± 0.02 | 0.1573 ± 0.02 | 0.1648 ± 0.02 | 0.1617 ± 0.01 | 0.1640 ± 0.02 |
| | Left testis | 0.0962 ± 0.01 | 0.1017 ± 0.01 | 0.0957 ± 0.02 | 0.1065 ± 0.01 | 0.1041 ± 0.01 | 0.1070 ± 0.01 |
| | Right testis | 0.1048 ± 0.01 | 0.1014 ± 0.01 | 0.0974 ± 0.01 | 0.1085 ± 0.01c* | 0.1052 ± 0.01c* | 0.1060 ± 0.01c* |
| | Pancreas | 0.2911 ± 0.06 | 0.2873 ± 0.03 | 0.2963 ± 0.04 | 0.3329 ± 0.05b* | 0.3115 ± 0.03 | 0.3202 ± 0.07 |
| | adipose tissue | 0.8329 ± 0.23 | 1.4797 ± 0.44a*** | 0.8636 ± 0.35b** | 1.2216 ± 0.41a*c* | 1.0369 ± 0.44b* | 1.0047 ± 0.30b* |
| 2G1 | Heart | 0.146 ± 0.018 | 0.120 ± 0.014 | 0.117 ± 0.010 | 0.128 ± 0.023 | 0.114 ± 0.010 a** | 0.121 ± 0.011a** |
| | Liver | 1.102 ± 0.056 | 1.317 ± 0.219 | 1.054 ± 0.089 | 1.290 ± 0.163 a*c** | 1.211 ± 0.114 c** | 1.293 ± 0.273 c* |
| | Spleen | 0.075 ± 0.006 | 0.087 ± 0.016 | 0.074 ± 0.013 | 0.107 ± 0.037 c* | 0.101 ± 0.026 c* | 0.095 ± 0.028 c* |
| | Lung | 0.147 ± 0.018 | 0.154 ± 0.033 | 0.140 ± 0.008 | 0.146 ± 0.010 | 0.139 ± 0.020 | 0.144 ± 0.019 |
| | Left kidney | 0.143 ± 0.020 | 0.169 ± 0.021 | 0.14 ± 0.015 b** | 0.156 ± 0.021 | 0.157 ± 0.030 | 0.166 ± 0.040 |
| | Right kidney | 0.138 ± 0.010 | 0.159 ± 0.017 a* | 0.135 ± 0.048 b* | 0.147 ± 0.018 | 0.139 ± 0.029 | 0.175 ± 0.063 |
| | Left testis | 0.102 ± 0.007 | 0.092 ± 0.004 a* | 0.096 ± 0.005 b* | 0.094 ± 0.037 | 0.100 ± 0.006 b** | 0.086 ± 0.017 e* |
| | Right testis | 0.100 ± 0.012 | 0.088 ± 0.032 | 0.095 ± 0.007 | 0.100 ± 0.021 b* | 0.095 ± 0.011 | 0.096 ± 0.011 |
| | Pancreas | 0.162 ± 0.015 | 0.144 ± 0.019 | 0.199 ± 0.050 | 0.129 ± 0.2a**c*** | 0.155 ± 0.033 c*d* | 0.145 ± 0.032 c* |
| | adipose tissue | 0.990 ± 0.223 | 1.107 ± 0.453 | 0.690 ± 0.292 b* | 1.202 ± 0.406 c** | 1.263 ± 0.276 c*** | 1.311 ± 0.629 c* |

Note:
P < 0.05*, 0.0 1*, 0.001;
a, b, c, d and e represent comparison with NaCl-PB group, model control group, Liraglutide group, L-dose group and M-dose group, respectively.

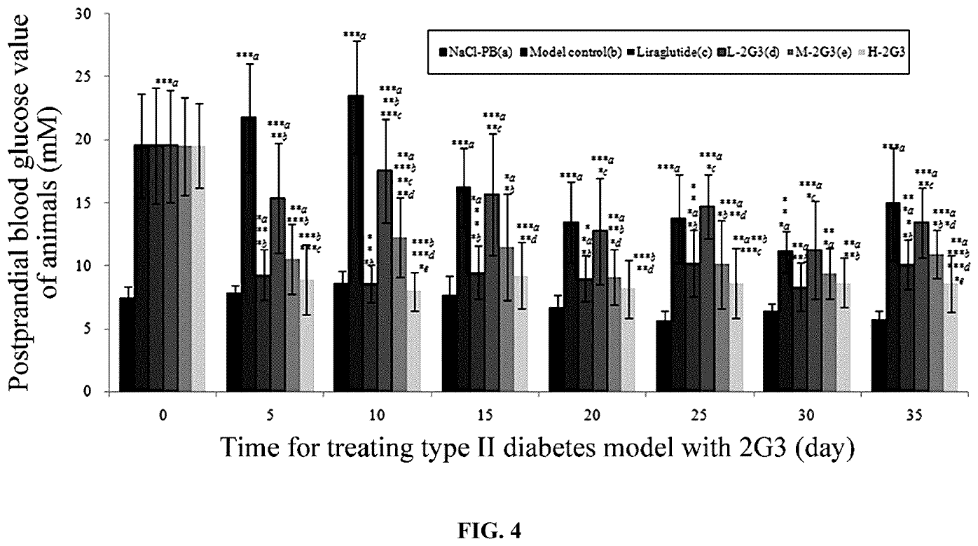

5. Hypoglycemic effect in T2D treatment: Compared with the NaCl-PB group, T2D model group had significant reduction in glycosylated hemoglobin (HbA1c) (P<0.01 or 0.001) and FPG (P<0.0 1), indicating that the T2D models were prepared successfully. Compared with the T2D model control group, fasting HbA1c reduction (−29%) (P<0.01) or FPG reduction (−50.2%) (P<0.01) in Liraglutide group was significant. The HbA1c reduction (−8, −23, −32% vs L-, M-, H-dose) (P<0.05 or 0.01) or FPG reduction (−26.3, −46.9, −47.3%) (P<0.01) in 2G3 group showed a dose-dependent manner. According to the results of dynamic PPG changes (FIG. 4), there was no significant difference in PPG before treatment in the T2D groups. After injection of Liraglutide or 2G3 peptide, the PPG level in the Liraglutide group significantly decreased. More administration resulted in better hypoglycemic effect. The PPG level in the 2G3 groups showed a dose-dependent decrease, and the change in blood glucose in the M-2G3 group was similar to that in Liraglutide group. In the 35-day T2D treatment, compared with Liraglutide group, the H-2G3 group had lowered PPG levels on the Day 5 and 25 (P<0.001), and the L-2G3 group had significantly higher PPG levels than the Liraglutide group on days 10-35 (P<0.05, 0.01, or 0.001). The PPG levels in the M-2G3 group on days 10, 20 and 25 and in the H-2G3 group on days 15 and 20 were lower than those in the L-2G3 group (P<0.05 or 0.01). PPG, or FPG, HbA1c had similar changes in the T2D treatment. 2G1 had no hypoglycemic effect on the T2DM model.
6. Measurement of blood biochemical parameters in the T2D treatment: In the experiment, there were significant changes in blood biochemical parameters (Table 4). The fasting insulin levels in the model control group (0.625±0.23 ng/ml) and the Liraglutide group (0.595±0.21 ng/ml) were much lower than that in the NaCl-PB group (1.411±3.01 ng/ml). The fasting insulin levels in the 2G3 groups showed a dose-dependent increase (0.626±0.23, 1.141±0.66 and 1.568±1.79 ng/ml), showing that the insulin level in the M- or H-2G3 group was increased by 2.38 times, which was obviously higher than those in the model control group, the Liraglutide group, and the L-2G3 group (P<0.05). The L- or H-2G3 group significantly had more platelets than the NaCl-PB group, or/and the model control group, and Liraglutide group (P<0.05 or 0.01). The Hb value in the H-2G3 group was lower than that in the NaCl-PB group (P<0.05), but had no effect on RBC and WBC. The Alanine aminotransferase (ALT), aspartate aminotransferase (AST), or alkaline phosphatase (ALP) in the 2G3 groups showed a dose-dependent decrease, but the ALP was significantly higher than that in Liraglutide group (P<0.01 or 0.001). The ALP or/and ALT levels in the M- or H-2G3 group were lower than those in the NaCl-PB group (P<0.05 or 0.01), and the AST or ALT level in the H-2D3 group was lower than that in the model control group (P<0.05). Compared with the NaCl-PB group, the albumin value in the T2D group was significantly decreased (P<0.001), but had dose-dependent increase with more 2G3 injection. The total cholesterol, high-density lipoprotein or low-density lipoprotein cholesterol in the T2D model group were significantly increased compared with those in the NaCl-PB group (P<0.001). Compared with the Liraglutide group, the total cholesterol and high-density lipoprotein cholesterol (HDL-C) in the 2G3 groups were all significantly increased (P<0.001 or 0.05). The total cholesterol or triglyceride in the Liraglutide group and the H-2G3 group were significantly lower than those in the model control group (P<0.05). Compared with the NaCl-PB group, the amylases in the model control group, the M-2G3 group, and the H-2G3 group were significantly increased (P<0.05 or 0.01).

The 2G1 groups showed a dose-dependent decrease in insulin (P>0.05). The ALT level in the L-2G1 group was higher than those in the NaCl-PB group and the Liraglutide group, and the ALT level in the M-2G1 group was lower than those in the model control group and the L-2G1 group (P<0.05 or 0.01). The AST level in the M-2G1 group was significantly lower than that in the L-2G1 group (P<0.05), and the AST level in the H-2G1 group was significantly higher than that in the M-2G1 group (P<0.05). Compared with the NaCl-PB group or the L-2G1 group, the ALP level in the M-2G1 group was lower (P<0.05). The 2G1 groups showed a dose-dependent decrease in albumin (P<0.05, 0.01 or 0.001), and the albumin in the model control group was significantly lower than that in the NaCl-PB group (P<0.05). The serum creatinine level in the 2G1 groups was lower than that in the NaCl-PB or Liraglutide group, and had a dose-dependent decrease (P<0.05, 0.01 or 0.001). The total cholesterol (T-CHO) or HDL-CHO in the 2G1 groups had an dose-dependent decrease, whereas the T-CHO or/and HDL-CHO and LDL-CHO levels in the Liraglutide group or the 2G1 groups were significantly higher than those in the NaCl-PB group (P<0.01 or 0.001). The T-CHO and HDL-C-CHO levels in the L- and M-2G1 groups were significantly higher than those in the Liraglutide group (P<0.05 or 0.01). As 2G3, 2G1 significantly promoted synthesis of HDL. The HDL-CHO level in the H-2G1 group was significantly lower than that in the model control group (P<0.05). There was no significant difference in triglyceride (TG) between the groups. Interestingly, compared with the NaCl-PB group, the amylase in the 2G1 groups had a dose-dependent-decrease (P<0.05 or 0.01), indicating an obvious protective effect on pancreatic exocrine cells (Table 4).

TABLE 4
Blood biochemical parameters of T2D model (mean ± SD, n ± 10)

| Dimer | Indicators | NaCl-PB | Control group | Liraglutide | L-2G3 | M-2G3 | H-2G3 |
|---|---|---|---|---|---|---|---|
| 2G3 | Insulin | 1.411 ± 3.01 | 0.625 ± 0.23 | 0.595 ± 0.21 | 0.626 ± 0.23 | 1.141 ± 0.66 | 1.568 ± 1.79 |
| fasting | (ng/ml) | | | | | b*c*d* | |
| serum | Leukocyte | 5.975 ± 1.95 | 6.528 ± 1.42 | 7.965 ± 1.91 b* | 6.475 ± 2.43 | 7.797 ± 1.97 | 7.085 ± 1.39 |
| | (10⁹/L) | | | | | | |
| | Erythtocyte | 10.115 ± 0.88 | 10.124 ± 0.81 | 10.157 ± 0.75 | 9.735 ± 0.67 | 9.936 ± 0.69 | 9.629 ± 0.52 |
| | (1012/L) | | | | | | |
| | Blood platelet | 1213.4 ± 124.72 | 1175.6 ± 169.14 | 1266.5 ± 242.80 | 1388.6 ± 270.85 a* | 1384.2 ± 283.78 | 1572.3 ± 323.66 |
| | (109/L) | | | | | | a**b**c* |
| | Hemoglobin | 146.8 ± 12.06 | 144.8 ± 11.90 | 144.2 ± 10.89 | 138.5 ± 9.30 | 141.2 ± 9.68 | 136.7 ± 7.27 a* |
| | (g/L) | | | | | | |
| | Glutamic- | 40.95 ± 12.60 | 38.84 ± 13.57 | 30.21 ± 8.23 a* | 40.70 ± 12.00 c* | 32.00 ± 5.92 | 26.37 ± 3.52 |
| | pyruvic- | | | | | | a**b*d**c* |
| | transainase | | | | | | |
| | (U/L) | | | | | | |
| | Glutamic | 141.04 ± 56.28 | 167.74 ± 30.38 | 144.57 ± 29.02 | 141.67 ± 31.23 | 140.01 ± 33.33 | 134.19 ± 27.95 b* |
| | oxalacetic | | | | | | |
| | transaminase | | | | | | |
| | (U/L) | | | | | | |
| | Alkaline | 86.33 ± 16.43 | 67.46 ± 13.68 a* | 55.82 ± 8.68 | 77.33 ± 16.95 c** | 73.06 ± 7.41 | 70.08 ± 6.92 |
| | phosphatase | | | a***b* | | a*c*** | a**c*** |
| | (U/L) | | | | | | |
| | Albumin | 34.54 ± 2.12 | 30.36 ± 1.74 a*** | 30.49 ± 1.03 a*** | 29.80 ± 1.13 a*** | 30.30 ± 1.10 | 30.59 ± 1.84 |
| | (g/L) | | | | | a*** | a*** |
| | Creatinine | 5.7 ± 1.77 | 5.0 ± 2.05 | 5.6 ± 1.58 | 5.6 ± 1.43 | 5.7 ± 1.34 | 6.6 ± 1.71 |
| | (μM) | | | | | | |
| | Total | 3.266 ± 0.43 | 4.969 ± 0.35 a*** | 4.684 ± 0.23 | 4.966 ± 0.34a***c* | 5.095 ± 0.22 | 4.928 ± 0.32 |
| | cholesterol | | | a***b* | | a***c*** | a*** |
| | (mM) | | | | | | |
| | Triglyceride | 1.075 ± 0.32 | 1.148 ± 0.29 | 1.001 ± 0.26 | 1.006 ± 0.23 | 1.046 ± 0.49 | 0.905 ± 0.17 b* |
| | fatty acid | | | | | | |
| | (mM) | | | | | | |
| | High-density | 2.638 ± 0.31 | 3.788 ± 0.31 a*** | 3.631 ± 0.18 a*** | 3.774 ± 0.22 a*** | 3.849 ± 0.21 | 3.839 ± 0.17 |
| | liporotein | | | | | a***c* | a***c* |
| | (mM) | | | | | | |
| | Low-density | 0.256 ± 0.08 | 0.540 ± 0.05 a*** | 0.509 ± 0.16 a*** | 0.562 ± 0.16 a*** | 0.594 ± 0.09 | 0.526 ± 0.12 |
| | liporotein | | | | | a*** | a*** |
| | (mM) | | | | | | |
| | Amylase | 3134.3 ± 342.20 | 3526.3 ± 357.76 a* | 3495.2 ± 481.92 | 3669.4 ± 1548.36 | 4054.6 ± 775.54 | 3682.7 ± 656.24 |
| | (U/L) | | | | | a** | a* |
| | Lipase (U/L) | 64.19 ± 39.96 | 45.79 ± 14.37 | 51.04 ± 11.66 | 48.50 ± 14.89 | 54.38 ± 21.77 | 46.28 ± 14.05 |
| | Glycosylated | 7.3 ± 2.45 | 20.0 ± 5.03 a*** | 14.2 ± 2.20 | 18.4 ± 4.33 | 15.4 ± 2.59 | 13.6 ± 2.22 |
| | hemoglobin | | | a***b** | a*** c* | a*** b* | a*** b** d** |
| | (mmol/ml) | | | | | | |
| | Fasting blood | 5.171 ± 4.24 | 14.149 ± 5.95 a** | 7.042 ± 1.63 b** | 10.423 ± 2.86 | 7.505 ± 1.64 | 7.454 ± 1.99 |
| | glucose | | | | a**c** | b**d* | b**d* |
| | (mM) | | | | | | |
| 2G1 | Insulin | 1.411 ± 3.01 | 0.625 ± 0.23 | 0.595 ± 0.21 | 0.818 ± 0.56 | 0.566 ± 0.23 | 0.477 ± 0.21 |
| post- | (ng/ml) | | | | | | |
| prandial | Alamine | 33.20 ± 2.96 | 67.81 ± 34.92 | 44.98 ± 21.18 | 91.77 ± 42.61 | 37.81 ± 12.45 | 58.84 ± 35.90 |
| serum | amino- | | | | a*c** | b*d** | |
| | transferase | | | | | | |
| | (U/L) | | | | | | |
| | Glutamic | 14170 ± 21.14 | 202.17 ± 175.72 | 124.69 ± 20.08 | 160.48 ± 52.92 | 118.60 ± 24.38 d* | 151.40 ± 40.15 e* |
| | oxalacetic | | | | | | |
| | transaminase | | | | | | |
| | (U/L) | | | | | | |
| | Alkaline | 92.98 ± 13.96 | 91.90 ± 20.08 | 97.52 ± 32.80 | 90.56 ± 15.54 | 75.716 ± 8.00 | 85.44 ± 17.29 |
| | phosphatase | | | | | a*d* | |
| | (U/L) | | | | | | |
| | Albumin | 36.18 ± 1.95 | 32.01 ± 2.20 a* | 34.11 ± 1.94 | 32.30 ± 2.32 a* | 30.78 ± 2.32 | 30.36 ± 2.08 |
| | (g/L) | | | | | a**c** | a***c*** |
| | Creatinine | 5.75 ± 2.87 | 3.33 ± 2.00 | 4.67 ± 1.41 | 2.90 ± 1.20 | 3.22 ± 1.56 a* | 3.10 ± 1.52 a*c* |
| | (μM) | | | | a*c** | | |
| | Total | 3.1 ± 0.37 | 5.72 ± 0.95 a*** | 4.94 ± 0.66 a*** | 5.76 ± 0.33 | 5.58 ± 0.49 | 5.39 ± 1.21a** |
| | choleterol | | | | a***c** | a*** c* | |
| | (mM) | | | | | | |
| | Triglyceride | 1.52 ± 0.48 | 1.72 ± 0.90 | 1.52 ± 0.75 | 1.75 ± 0.76 | 1.88 ± 1.81 | 1.707 ± 1.13 |
| | fatty acid | | | | | | |
| | (mM) | | | | | | |
| | Lipoprotein | 1.37 ± 0.19 | 2.37 ± 0.29 a*** | 2.02 ± 0.18 | 2.29 ± 0.19 | 2.18 ± 0.17 | 2.03 ± 0.37 |
| | | | | a***b** | a***c** | a*** | a**b* |
| | Lipoprotein | 0.30 ± 0.05 | 0.40 ± 0.12 | 0.42 ± 0.06 a** | 0.43 ± 0.10 a* | 0.44 ± 0.10 a* | 0.434 ± 0.18 |
| | Amylase | 1826.8 ± 312 | 1440.9 ± 251 a* | 1609.1 ± 122 | 1493.2 ± 184 a* | 1376.7 ± 180 a** | 1247.8 ± 265 |
| | (U/L) | | | | | | d* a** |
| | Postprandial | 7.6 ± 1.5 | 26.2 ± 4.6 a*** | 18.4 ± 10.3 a* | 25.4 ± 2.7 a *** | 26.3 ± 2.8 | 24.7 ± 4.6 |
| | blood | | | | | c*a*** | a*** |
| | glucose | | | | | | |
| | (mM) | | | | | | |

Note:
P < 0.05*, 0.01*, 0.001***;
a, b, c, d and e were compared with the model control group, the Liraglutide, L-, M- and H-dose groups, respectively.

### Example 4 Tissue Pathological Analysis of T2D Model Treated by the Dimers

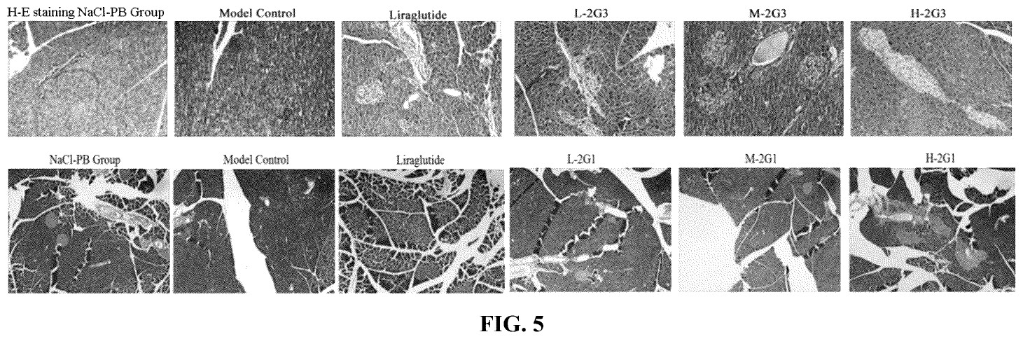

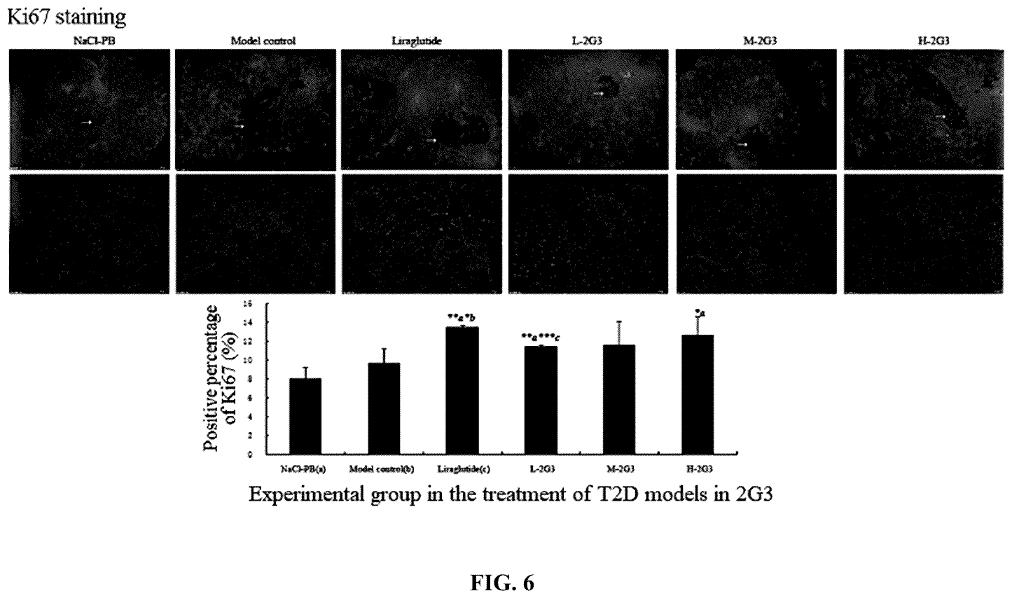

1. H-E staining: The pancreatic tissue in the T2D model showed sparse pancreatic acinus with obvious pyknosis and many pathological vacuoles. The pancreatic islet cells in the model control group underwent deformation, atrophy and pyknosis. The acinar cells in the Liraglutide group showed strong eosinophilic staining with enlarged inter-cellular spaces. The acinar cells in the 2G3 or 2G1 groups had dense and no pathological vacuoles compared with NaCl-PB group (FIG. 5).
2. Fluorescent staining of Ki67 protein: The anti-Ki67 antibody was used to obtain the distribution and localization of Ki67 protein in the pancreatic tissue of the T2D model. For the NaCl-PB group, scattered positive acinar cells were observed around pancreatic islets, or acinar cells near the ducts, in the ductal epithelium. For the model control group, there were many positive acinar cells around pancreatic islets and exocrine cells, such as ducts and acinar cells. In the Liraglutide group, lobular acinar cells showed scattered positive cells in pancreatic islets and no positive ductal epithelial cells were observed. The Ki67 protein in the Liraglutide group was significantly higher than those in the NaCl-PB group or the model control group (P<0.05). The Ki67 protein in the 2G3 groups had a dose-dependent increase. Compared with the NaCl-PB group, the Ki67 protein in the L- or H-2G3 group significantly increased (P<0.05), and there is a significant difference between the L-2G3 and the Liraglutide group (P<0.001), indicating that 2G3 significantly promoted the proliferation of pancreas or pancreatic islet cells (FIG. 6).

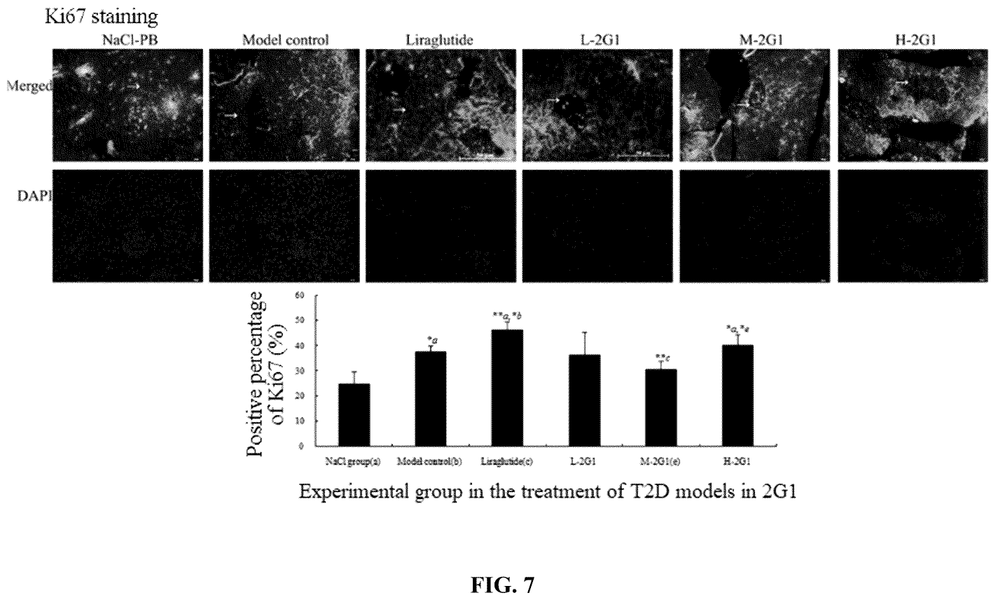

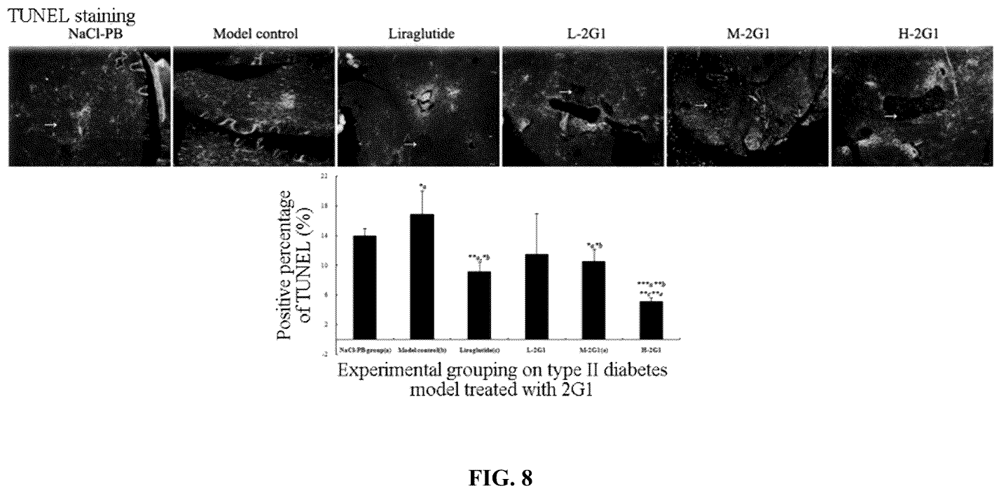

The Ki67 protein in the model control, the Liraglutide, and the H-2G1 groups was significantly higher than that in the NaCl-PB group (P<0.05 or 0.01). Compared with the model control or the M-2G1 group, the Liraglutide and the H-2G1 group had significant differences (P<0.05). The Ki67 expression in the M-2G1 group was lower than that in the Liraglutide group (P<0.01). These results revealed that 2G1 significantly promoted the proliferation of pancreatic cells (FIG. 7).
3. TUNEL staining: For the model control group, a large number of positive cells were observed in the lobular acinus and ductal epithelium. Scattered pancreatic islets and some pancreatic islet-positive cells were observed in the pancreatic tissue. For the Liraglutide group, there were obvious positive cells in the lobular acinus, Scattered positive cells were observed in the pancreatic islets, but no or less positive ductal cells were observed. In the 2G1 groups, there were few or scattered positive lobular cells and there were few or no positive ductal cells. The 2G1 groups showed a dose-dependent decrease in TUNEL. The Liraglutide, M-2G1, H-2G1 groups had significantly lower positive rate than those of the NaCl-PB group or the model control group (P<0.05, 0.01 or 0.001). The H-2G1 group had a lower positive rate in TUNEL than those in the Liraglutide or the M-2G1 group (P<0.01) (FIG. 8). These results revealed that the 2G1 peptide obviously protected the apoptosis of pancreatic cells. Each of the 2G3 groups did not show TUNEL positive change.

### Example 5 Analysis of Glucagon-Like Peptide-1 Receptor (GLP-1R)

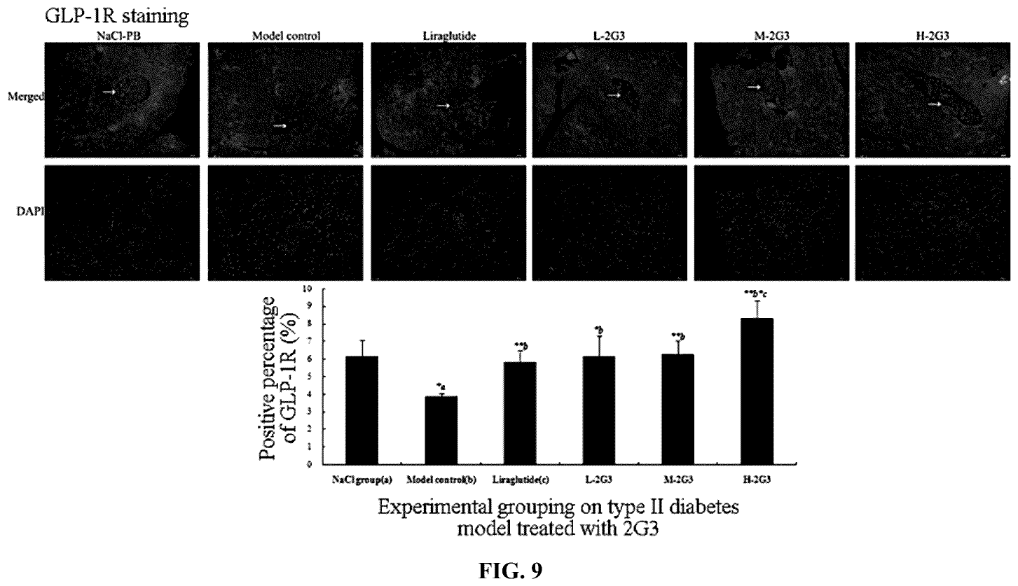

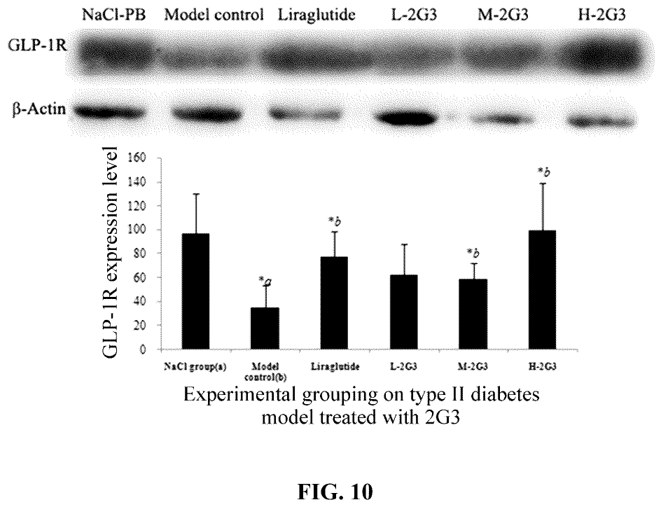

1. Immunohistochemical (IHC) staining: GLP-1R in the 2G3 groups had an dose-dependent increase. Compared with the model control group, both of the Liraglutide group and the 2G3 groups had significantly increased GLP-1R (P<0.05 and 0.01). The expression of GLP-1R in the H-2G3 group was significantly higher than that in the Liraglutide group, and the expression of GLP-1R in the model control group was lower than that in the NaCl-PB group (P<0.0 5) (FIG. 9)
2. Western blot analysis: Compared with the model control group, the Liraglutide, L-2G2, or H-2G3 group had significantly increased GLP-1R (P<0.05). The expression of GLP-1R in the model control group was lower than that in the NaCl-PB group (P<0.05) (FIG. 10).

### Example 6 Insulin IHC Analysis

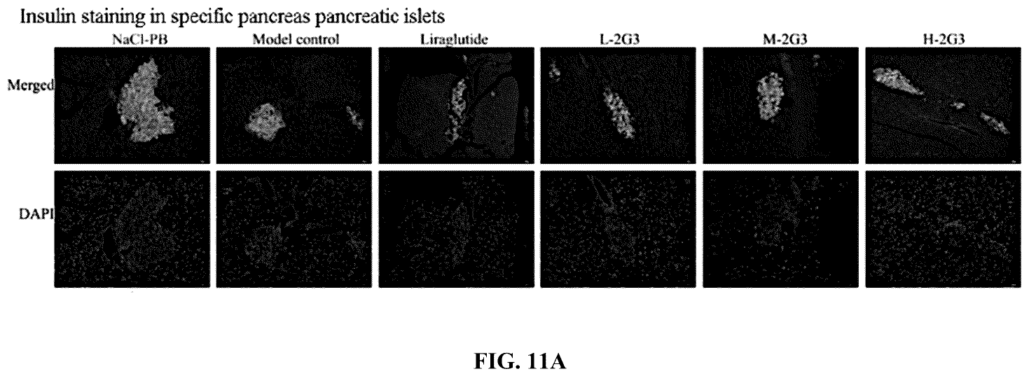

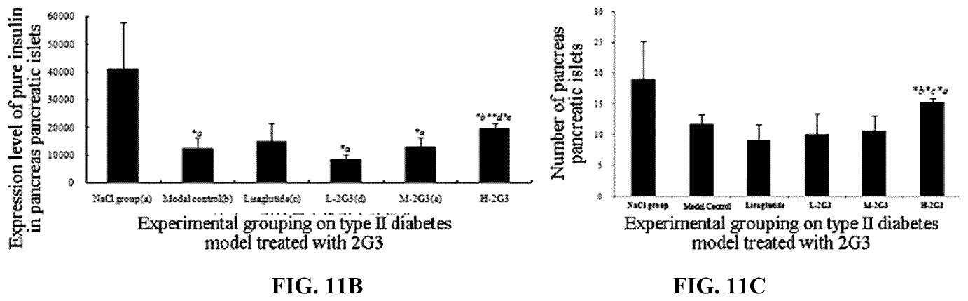

Distribution and location of insulin in T2D pancreatic islets were observed by using an anti-insulin antibody (FIGS. 11A-11C). The insulin expressions of pancreatic islets in the model control group and the 2G3 groups were lower than that in the NaCl-PB group (P<0.05). The insulin staining intensity and the number of pancreatic islets in the 2G3 groups were increased on a dose-dependent manner (P<0.05 or 0.01).

Conclusions: From the above examples, concluded that the classification based on the effective duration clearly distinguishes the characteristics of long-acting and short-acting moleculars. Obviously, the homodimeric 2G3 and 2G6 series developed belong to the longest-acting molecules. The 2G3 peptide as a representative in dimeric peptides induces the synthesis of insulin by binding with GLP-1R and generate the hypoglycemic effect in T2D model. The biological effects of active GLP-1 homodimers were evaluated in various assays. These studies suggest that the dimeric peptides show the most promising application for T2D in rodent models, such as the longest-lasting hypoglycemic effects and other effects in weight loss and organ protection.

The structure-activity relationship reveals that the dimers without Aib have the best solubility in water and the dimers with Aib or even an amidated moiety at the C-terminus have a poor solubility in water. The individual peptide of them can maintain a long activity. These properties suggest that in the 2G3 peptide, the N-terminal moiety containing ⁸Ala sequence may be wrapped by the symmetrical ²⁶Lys-glutamyl fatty acid chain in the dimer to form a hydrophobic core, which in turn is surrounded by a hydrophilic polypeptide chain and is not easily hydrolyzed by DPP-4, so that a longer effect is maintained. For the sequences containing Aib or even having an amidated moiety at the C-terminus, the Aib and the amidated moiety may be exposed, resulting in low solubility in water. Since Aib is not a substrate for DDP-4, it can maintain a longer activity. Aminoisobutyric acid (Aib) and β-Ala are similar to L-α-Ala or Gly, β-Aib and β-Ala are also normal metabolites of human pyrimidine nucleotides, and can be highly tolerated in human body. Therefore, the toxic reaction of these compounds should be extremely low, and thus, the present disclosure uses these amino acids substitution to significantly prolong the hypoglycemic activity.

In the hypoglycemic effect of normal mice, the results of single OGTT experiment showed that the dimer has a long hypoglycemic effect through slow absorption in the blood. The results of the multiple OGTT experiment showed that the long duration effect involves the amino acid at position 8 of the polypeptide, the position of the disulfide bond in the dimer, the symmetrical ²⁶Lys fatty acid modification and the C-terminal amidation, and is independent of the Lys modification at multiple sites in the same molecule. Table 2 shows that the long active structure contains ⁸Aib, ¹⁸Cys-Cys disulfide bond, symmetrical oleoyl-1-γ-glutamoyl-²⁶Lys, and C-terminal amidated moiety. These modifications are characterized in that (1) α-/β-Aib or β-Ala→⁸Ala substitution produces longer activity, in which α-Aib substitution generates the best effect; (2) compared with other fatty acid modifications, the mono-oleoyl-L-γ-glutamyl-²⁶Lys structure achieves the best results; (3) C-terminal amidation significantly prolongs the activity: (4) the disulfide bond moiety at the position 18 in the dimer molecule shows the best activity; (5) PEG modification significantly shortens the specific activity (hypoglycemic duration per mg) while prolonging the half-life; (6) the activity of the monomeric peptide is only ½-¼ of that of the corresponding dimer.

In the T2D treatment experiments, the HbA1c reduction (−8, −23, −32%) or FPG reduction (−26.3, −46.9 and −47.3%) in the 2G3 groups and the fasting HbA1c reduction (−29%) or FPG reduction (−50.2%) in the Liraglutide group obviously showed hypoglycemic effects, indicating that 2G3 peptides and Liraglutide in the same molar concentration have similar effects on PPG, FPG and HbA1c.

The body weight of 2G3 groups had a dose-dependent decrease. The H-2G3 group had similar weight curve in body weight or adipose tissue to that in Liraglutide group, suggesting that it had less influence on diet and fat metabolism compared with Liraglutide. This was also confirmed by statistic data in drinking water or food during the preparation of T2D animals. However, the weights of some organs such as left kidney, right testis, and adipose tissue increased, indicating that compared with Liraglutide, this dimer has less influence on diet and fat metabolism. 2G3 caused the increase of liver weight and alanine aminotransferase, aspartate aminotransferase and alkaline phosphatase showed a dose-dependent decrease, indicating that the drug has a strong protective effect on liver and heart. But 2G3 led to a higher alkaline phosphatase level than Liraglutide, indicating stronger stimulation to liver. The increase in platelet quantity and spleen weight showed that 2G3 can promote hemostasis to protect the integrity of the vascular wall in T2D models. The Albumin in the 2G3 groups had a dose-dependent increase, suggesting that 2G3 may be transported by binding to albumin like Liraglutide. However, compared with the normal NaCl-PB group, the T2D model groups had significant decrease in albumin, showing that the hypertension, hyperlipidemia, hyperglycemia, or STZ reagent caused less albumin. 2G3 induced more total cholesterol, low-density lipoprotein cholesterol, and high-density lipoprotein cholesterol, showing that it increase the synthesis of cholesterol. Compared with the Liraglutide group, the total cholesterols were higher in the L- and M-2G3 groups, and the high-density lipoproteins was higher in the M- and H-2G3 groups, indicating that 2G3 promotes the retrograde transport of cholesterol by increasing the high-density lipoprotein. The significant pancreatic enlargement and higher amylase level in the M- and H-2G3 groups indicated that 2G3 had a certain promoting effect on pancreatic exocrine. 2G3 had no effects on kidney and lung function as well as white blood cells, red blood cells, hemoglobin, creatinine, and triglyceride.

The 2G1 groups showed significant increase in weights of liver and spleen, higher alanine aminotransferase, and aspartate aminotransferase levels, and less alkaline phosphatase and albumin levels, indicating that it significantly affects the functions of liver and spleen.

In the T2D treatment experiment with 2G3, the NaCl-PB mice (HbA1c 7.3±2.45 mm and FPG 5.171±4.24 mm) had the normal insulin level (1.411±3.01 ng/ml) and T2D control mice (HbA1c 20±5.03 mm and FPG 14.149±5.95 mm) had the T2D insulin value (0.625±0.23 ng/ml), but the Liraglutide group (HbA1c 14.2±2.20 mm and FPG 7.042±1.63 mm) induced the insulin value of 0.595±0.21 ng/ml, showing that T2D showed significant increase in insulin tolerance. Meanwhile Liraglutide induced a lower insulin level by the inhibition of diet. The insulin content (0.626±0.23, 1.141±0.66, 1.568±1.79 ng/ml) in the 2G3 groups had a dose-dependent increase. The percentage increments of these insulin values corresponding to the Liraglutide group are +5.2, +91.8, and +163.5%, indicating that 2G3 has stronger insulin release than Liraglutide, and therefore 2G3 has a better hypoglycemic effect. If the hypoglycemic effect was evaluated according to the secretion amount of insulin, the L-2G3 group should have a bioequivalent relationship with the Liraglutide group, and the hypoglycemic effect of the M- or H-2G3 group should be doubled or higher, but the M-2G3 group actually shows similar hypoglycemic effect with the Liraglutide group, which reflects that higher dose of 2G3 does not induce a greater hypoglycemic effect, or even hypoglycemia when the blood glucose level was normal. In this experiment, 8 and 68 times of low dose of 2G3 did not induce any one of 6 KM mice fasted for 13 hour to produce hypoglycemia within 3 hours after the administration, indicating that the dimeric peptides do not induce hypoglycemia.

The results of H-E staining showed that compared with the NaCl-PB group, 2G3 or 2G1 strongly protected pancreatic acinar cells and rescued pathological damages in T2D models, such as sparse acini, pathological vacuoles, pancreatic islet cell deformation, atrophy or pyknosis. 2G3 induced a dose-dependent increase in Ki67, suggesting that 2G3 promotes the proliferation of pancreatic cells. The expression of Ki67 in the M-2G1 group was lower than that in the Liraglutide group, indicating that 2G1 group had a weaker proliferation of pancreatic cells than the Liraglutide group. The TUNEL positive rate in the 2G1 groups had a dose-dependent decrease, and the TUNEL positive rate in the H-2G1 group was lower than that in the Liraglutide group or the M-2G1 group, indicating that 2G1 significantly protects pancreatic cells such as acinus and ducts against STZ toxicity or pathological damage. 2G3 induced significant increase in GLP-1R expression, the insulin staining intensity, and the number of pancreatic islet, suggesting that the hypoglycemic effect of 2G3 was mediated by GLP-1R, resulting in more insulin release and more pancreatic islets.

Our conclusion is that the monomeric or dimeric peptides claimed in the present disclosure can induce more insulin release by binding to GLP-1R, thereby resulting in different hypoglycemic or pancreatic protective effects.

The above embodiments only represent several embodiments of the present disclosures, The descriptions are specific and detailed, but cannot be understood as a limitation in the scope of the present disclosures. It was noted that the persons skilled in the arts can make development and improvements in the concept of the present disclosures. These development and improvements fall into the protection scope of the present disclosures. Therefore, the protection scope of the present application shall be determined by the appended claims.

What is claimed is:

1. A monomeric glucagon-like peptide 1 analogue, comprising an amino acid sequence selected from the group consisting of the following four sequences:
(1) His-X₈-Glu-Gly-Thr-Phe-Thr-Cys-Asp-Val-Ser-Ser-Tyr-Leu-Glu-Gly-Gln-Ala-Ala-X₂₆-Glu-Phe-Ile-Ala-Trp-Leu-Val-X₃₄-X₃₅-Arg-X₃₇, as set forth in SEQ ID NO: 1;
(2) His-X₈-Glu-Gly-Thr-Phe-Thr-Ser-Asp-Val-Cys-Ser-Tyr-Leu-Glu-Gly-Gln-Ala-Ala-X₂₆-Glu-Phe-Ile-Ala-Trp-Leu-Val-X₃₄-X₃₅-Arg-X₃₇, as set forth in SEQ ID NO: 2;
(3) His-X₈-Glu-Gly-Thr-Phe-Thr-Ser-Asp-Val-Ser-Cys-Tyr-Leu-Glu-Gly-Gln-Ala-Ala-X₂₆-Glu-Phe-Ile-Ala-Trp-Leu-Val-X₃₄-X₃₅-Arg-X₃₇, as set forth in SEQ ID NO: 3; and
(4) His-X₈-Glu-Gly-Thr-Phe-Thr-Ser-Asp-Val-Ser-Ser-Tyr-Leu-Glu-Gly-Gln-Ala-Ala-X₂₆-Glu-Phe-Ile-Ala-Trp-Leu-Val-X₃₄-X₃₅-Arg-Gly-Cys-OH, as set forth in SEQ ID NO: 4;
wherein, X₈ is L-α-alanine, β-alanine, α-aminoisobutyric acid, or β-aminoisobutyric acid;
X₂₆ is lysine, lysine modified with alkanoylglutamyl on a side chain ε-amino, or lysine modified with an alkanoyl on the side chain ε-amino;
X₃₄ is Arg, Lys, or lysine modified with alkanoylglutamyl on the side chain ε-amino;
X₃₅ is Gly, Ala, β-alanine, α-amino isobutyric acid, or β-amino isobutyric acid;
X₃₇ is a moiety of Gly-COOH, Gly-NH₂, NH₂, or OH; or an allosteric amino acid sequence of a first 7-36 positions as provided in the above formed in a copy of a repeat sequence, wherein the X₈ in the repeat sequence is replaced in a glycine, or α-/β-aminoiso butyric acid, and cysteine is replaced in serine or glycine, the X₂₆ in the repeat sequence is arginine; or is PEG-modified by linking a C-terminal amido with a polyethylene glycol molecule (PEG), wherein a molecular weight of the PEG is 0.5-30 KD.

2. The monomeric glucagon-like peptide 1 analogue according to claim 1, wherein when the X₂₆ is the lysine modified with the alkanoylglutamyl [γ-Glu(N-α-alkanoyl)] on the side chain ε-amino, the monomeric glucagon-like peptide 1 analogue has a structural formula in Formula 1; or when the X₂₆ is the lysine modified with the alkanoyl on the side chain ε-amino, the monomeric glucagon-like peptide 1 analogue has a structural formula in Formula 2; in each of the Formulas 1 and 2, n is equal to 14 or 16;

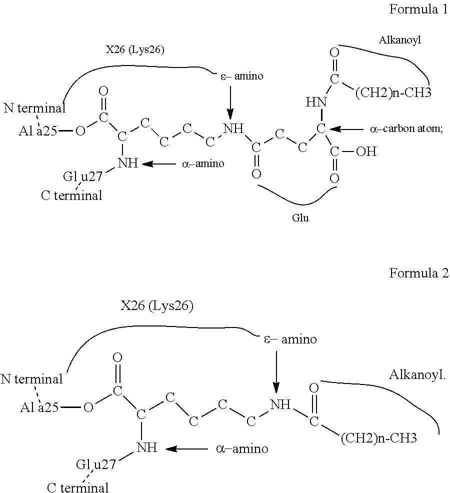

3. A glucagon-like peptide 1 analogue homodimer, wherein the glucagon-like peptide 1 analogue homodimer is formed by two identical monomers according to claim 2 through a disulfide bond formed by two cysteines, wherein the glucagon-like peptide 1 analogue homodimer is H- or U-like homodimer, and the glucagon-like peptide 1 analogue homodimer has an amino acid sequence selected from the group consisting of the following four sequences: 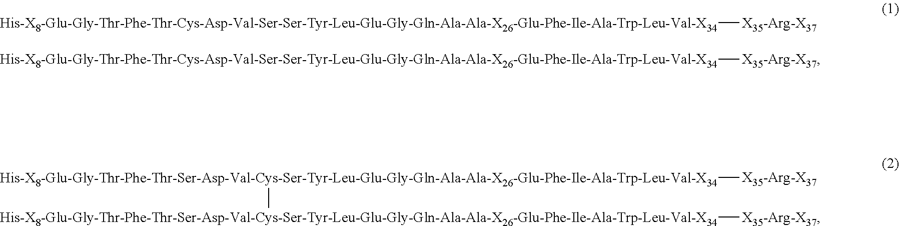 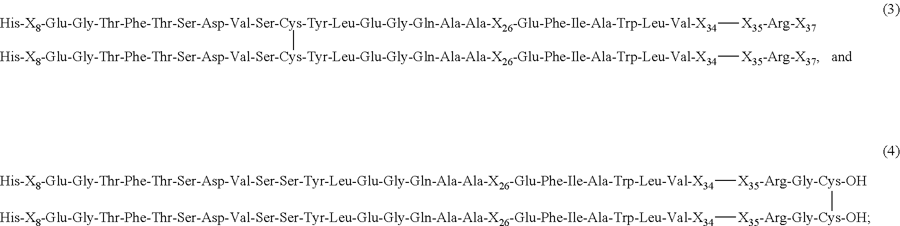
wherein, X₈ is L-α-alanine, β-alanine, α-amino isobutyric acid, or β-amino isobutyric acid;
X₂₆ is lysine, lysine modified with alkanoylglutamyl on a side chain ε-amino, or lysine modified with an alkanoyl on the side chain ε-amino;
X₃₄ is Arg, Lys, or lysine modified with alkanoylglutamyl on the side chain ε-amino;
X₃₅ is Gly, Ala, β-alanine, α-amino isobutyric acid, or β-amino isobutyric acid;
X₃₇ is a moiety of Gly-OH, Gly-NH₂, NH₂, or OH; or an allosteric amino acid sequence of the first 7-36 positions formed a copy of a repeat sequence, wherein the X₈ in the repeat sequence is replaced in a glycine, or α- or β-aminoisobutyric acid (Aib), the cysteine is replaced with serine or glycine, and the X₂₆ in the repeat sequence is arginine; or is PEG-modified by linking the C-terminal amido with a polyethylene glycol molecule (PEG), wherein the molecular weight of the PEG is 0.5-30 KD.

4. The glucagon-like peptide 1 analogue homodimer according to the claim 3, wherein when the X₂₆ is the lysine modified with the alkanoylglutamyl [γ-Glu(N-α-alkanoyl)] on the side chain ε-amino, the glucagon-like peptide 1 analogue homodimer has a structural formula in the Formula 1; or when the X₂₆ is the lysine modified with the alkanoyl on the side chain ε-amino, the glucagon-like peptide 1 analogue homodimer has a structural formula in the Formula 2; in each of the Formulas 1 and 2, n is equal to 14 or 16.

5. A method a preparation of a pancreas protective drug or/and a hypoglycemic drug for treating type II diabetes, comprising a step of using the monomeric glucagon-like peptide 1 analogue according to claim 1 preparation.

6. A drug for protecting pancreas or treating type II diabetes, wherein the monomeric glucagon-like peptide 1 analogue according to claim 1 is used as an active content of the drug.

7. The method according to claim 5, wherein wherein when the X₂₆ is the lysine modified with the alkanoylglutamyl [γ-Glu(N-α-alkanoyl)] on the side chain ε-amino, the monomeric glucagon-like peptide 1 analogue has a structural formula in Formula 1; or when the X₂₆ is the lysine modified with the alkanoyl on the side chain ε-amino, the monomeric glucagon-like peptide 1 analogue has a structural formula in Formula 2; in each of the Formulas 1 and 2, n is equal to 14 or 16;

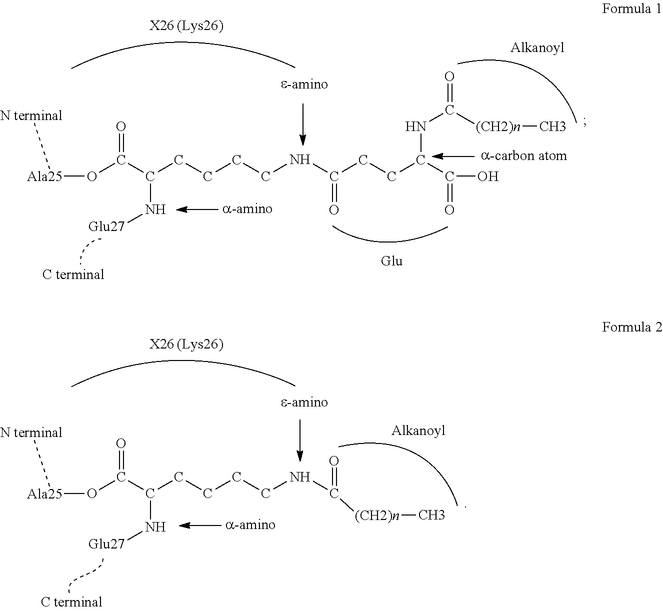

8. A method a preparation of a pancreas protective drug or/and a hypoglycemic drug for treating type II diabetes, comprising a step of using the glucagon-like peptide 1 analogue homodimer according to claim 3 in the preparation.

9. The method according to claim 8, wherein when the X₂₆ is the lysine modified with the alkanoylglutamyl [γ-Glu(N-α-alkanoyl)] on the side chain ε-amino, the glucagon-like peptide 1 analogue homodimer has a structural formula in the Formula 1; or when the X₂₆ is the lysine modified with the alkanoyl on the side chain ε-amino, the glucagon-like peptide 1 analogue homodimer has a structural formula in the Formula 2; in each of the Formulas 1 and 2, n is equal to 14 or 16.

10. The drug according to claim 6, wherein wherein when the X₂₆ is the lysine modified with the alkanoylglutamyl [γ-Glu(N-α-alkanoyl)] on the side chain ε-amino, the monomeric glucagon-like peptide 1 analogue has a structural formula in Formula 1; or when the X₂₆ is the lysine modified with the alkanoyl on the side chain ε-amino, the monomeric glucagon-like peptide 1 analogue has a structural formula in Formula 2; in each of the Formulas 1 and 2, n is equal to 14 or 16;

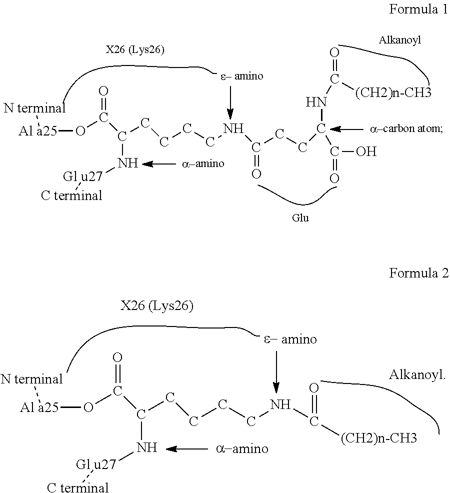

11. A drug for protecting pancreas or treating type II diabetes, wherein the glucagon-like peptide 1 analogue homodimer according to claim 3 is used as an active content of the drug.

12. The drug according to claim 11, wherein when the X₂₆ is the lysine modified with the alkanoylglutamyl [γ-Glu(N-α-alkanoyl)] on the side chain ε-amino, the glucagon-like peptide 1 analogue homodimer has a structural formula in the Formula 1; or when the X₂₆ is the lysine modified with the alkanoyl on the side chain ε-amino, the glucagon-like peptide 1 analogue homodimer has a structural formula in the Formula 2; in each of the Formulas 1 and 2, n is equal to 14 or 16.


SEQUENCE LISTING (71 sequences)

SEQ ID NO: 1 - protein, 31 aa, Artificial Sequence
  Sequence is synthesized
  misc_feature (2)..(2): Xaa is L-a-alanine (Ala), a-alanine (b-Ala), or a- / b-amino isobutyric acid (a- or b-Aib)
  misc_feature (20)..(20): Xaa is lysine, lysine modified with alkanoylglutamyl on the side chain e-amino, or lysine modified with an alkanoyl on the side chain e-amino
  misc_feature (28)..(28): Xaa is Arg, Lys, or lysine modified with alkanoylglutamyl on the side chain e-amino
  misc_feature (29)..(29): Xaa is Gly, Ala, b-alanine, a-amino isobutyric acid or b-amino isobutyric acid
  misc_feature (31)..(31): Xaa is a moiety of Gly-COOH (C-ternimal Gly with carboxyl group), Gly-NH2 (C-terminal Gly with amidation), NH2 (36Arg with amidation), or OH (36Arg with carboxyl terminus); or the allosteric amino acid sequence of the first 7-36 positions
HXEGTFTCDVSSYLEGQAAXEFIAWLVXXRX

SEQ ID NO: 2 - protein, 31 aa, Artificial Sequence
  Sequence is synthesized
  misc_feature (2)..(2): Xaa is L-a-alanine (Ala), a-alanine (b-Ala), or a- / b-amino isobutyric acid (a- or b-Aib)
  misc_feature (20)..(20): Xaa is lysine, lysine modified with alkanoylglutamyl on the side chain e-amino, or lysine modified with an alkanoyl on the side chain e-amino
  misc_feature (28)..(28): Xaa is Arg, Lys, or lysine modified with alkanoylglutamyl on the side chain e-amino
  misc_feature (29)..(29): Xaa is Gly, Ala, b-alanine, a-amino isobutyric acid or b-amino isobutyric acid
  misc_feature (31)..(31): Xaa is a moiety of Gly-COOH (C-ternimal Gly with carboxyl group), Gly-NH2 (C-terminal Gly with amidation), NH2 (36Arg with amidation), or OH (36Arg with carboxyl terminus); or the allosteric amino acid sequence of the first 7-36 positions
HXEGTFTSDVCSYLEGQAAXEFIAWLVXXRX

SEQ ID NO: 3 - protein, 31 aa, Artificial Sequence
  Sequence is synthesized
  misc_feature (2)..(2): Xaa is L-a-alanine (Ala), a-alanine (b-Ala), or a- / b-amino isobutyric acid (a- or b-Aib)
  misc_feature (20)..(20): Xaa is lysine, lysine modified with alkanoylglutamyl on the side chain e-amino, or lysine modified with an alkanoyl on the side chain e-amino
  misc_feature (28)..(28): Xaa is Arg, Lys, or lysine modified with alkanoylglutamyl on the side chain e-amino
  misc_feature (29)..(29): Xaa is Gly, Ala, b-alanine, a-amino isobutyric acid or b-amino isobutyric acid
  misc_feature (31)..(31): Xaa is a moiety of Gly-COOH (C-ternimal Gly with carboxyl group), Gly-NH2 (C-terminal Gly with amidation), NH2 (36Arg with amidation), or OH (36Arg with carboxyl terminus); or the allosteric amino acid sequence of the first 7-36 positions
HXEGTFTSDVSCYLEGQAAXEFIAWLVXXRX

SEQ ID NO: 4 - protein, 32 aa, Artificial Sequence
  Sequence is synthesized
  misc_feature (2)..(2): Xaa is L-a-alanine (Ala), a-alanine (b-Ala), or a- / b-amino isobutyric acid (a- or b-Aib)
  misc_feature (20)..(20): Xaa is lysine, lysine modified with alkanoylglutamyl on the side chain e-amino, or lysine modified with an alkanoyl on the side chain e-amino
  misc_feature (28)..(28): Xaa is Arg, Lys, or lysine modified with alkanoylglutamyl on the side chain e-amino
  misc_feature (29)..(29): Xaa is Gly, Ala, b-alanine, a-amino isobutyric acid or b-amino isobutyric acid
  misc_feature (32)..(32): C-terminal
HXEGTFTSDVSSYLEGQAAXEFIAWLVXXRGC

SEQ ID NO: 5 - protein, 31 aa, Artificial Sequence
  Sequence is synthesized
  misc_feature (20)..(20): K is 26Lys[N-e-(N-a-palmitoyl-L-g-glutamyl)]
  misc_feature (31)..(31): C-terminal
HAEGTFTSDVSSYLEGQAAXEFIAWLVRGRG

SEQ ID NO: 6 - protein, 32 aa, Artificial Sequence
  Sequence is synthesized
  misc_feature (20)..(20): Xaa is 26Lys[N-e-(N-a-Palmitoyl-g-glutamyl)]
  misc_feature (32)..(32): C-terminal
HAEGTFTSDVSSYLEGQAAXEFIAWLVRGRGC

SEQ ID NO: 7 - protein, 31 aa, Artificial Sequence
  Sequence is synthesized
  misc_feature (20)..(20): Xaa is Xaa is 26Lys[N-e-(N-a-Palmitoyl-g-glutamyl)]
  misc_feature (31)..(31): C-terminal
HAEGTFTSDVCSYLEGQAAXEFIAWLVRGRG

SEQ ID NO: 8 - protein, 31 aa, Artificial Sequence; identical to SEQ ID NO: 7
  Sequence is synthesized
  misc_feature (20)..(20): Xaa is Xaa is 26Lys[N-e-(N-a-Palmitoyl-g-glutamyl)]
  misc_feature (31)..(31): N-terminal

SEQ ID NO: 9 - protein, 31 aa, Artificial Sequence; identical to SEQ ID NO: 7
  Sequence is synthesized
  misc_feature (20)..(20): Xaa is 26Lys[N-e-(N-a-oleoyl-L-g-glutamyl)]
  misc_feature (31)..(1): C-terminal

SEQ ID NO: 10 - protein, 31 aa, Artificial Sequence; identical to SEQ ID NO: 7
  Sequence is synthesized
  misc_feature (20)..(20): Xaa is 26Lys[N-e-(N-a-oleoyl-L-g-glutamyl)]
  misc_feature (31)..(31): N-terminal

SEQ ID NO: 11 - protein, 31 aa, Artificial Sequence
  Sequence is synthesized
  misc_feature (2)..(2): Xaa is aAib
  misc_feature (20)..(20): Xaa is 26Lys[N-e-(N-a-Palmitoyl-g-glutamyl)]
  misc_feature (31)..(31): C-terminal
HXEGTFTSDVCSYLEGQAAXEFIAWLVRGRG

SEQ ID NO: 12 - protein, 31 aa, Artificial Sequence; identical to SEQ ID NO: 11
  Sequence is synthesized
  misc_feature (2)..(2): Xaa is aAib
  misc_feature (20)..(20): Xaa is 26Lys[N-e-(N-a-Palmitoyl-g-glutamyl)]
  misc_feature (31)..(31): N-terminal

SEQ ID NO: 13 - protein, 31 aa, Artificial Sequence; identical to SEQ ID NO: 11
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): N-terminal

SEQ ID NO: 14 - protein, 31 aa, Artificial Sequence; identical to SEQ ID NO: 11
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): C-terminal

SEQ ID NO: 15 - protein, 31 aa, Artificial Sequence; identical to SEQ ID NO: 11
  Sequence is synthesized
  misc_feature (2)..(2): Xaa is bAib
  misc_feature (20)..(20): Xaa is 26Lys[N-e-(N-a-oleoyl-L-g-glutamyl)]
  misc_feature (31)..(31): N-terminal

SEQ ID NO: 16 - protein, 30 aa, Artificial Sequence
  Sequence is synthesized
  misc_feature (2)..(2): Xaa is bAib
  misc_feature (29)..(29): Xaa is bAib
  misc_feature (30)..(30): N-terminal
HXEGTFTSDVCSYLEGQAAKEFIAWLVRXR

SEQ ID NO: 17 - protein, 30 aa, Artificial Sequence
  Sequence is synthesized
  misc_feature (2)..(2): Xaa is bAib
  misc_feature (29)..(29): Xaa is bAib
  misc_feature (30)..(30): N-terminal
HXEGTFTSDVCSYLEGQAAKEFIAWLVKXR

SEQ ID NO: 18 - protein, 31 aa, Artificial Sequence; identical to SEQ ID NO: 11
  Sequence is synthesized
  misc_feature (2)..(2): Xaa is bAla
  misc_feature (20)..(20): Xaa is 26Lys[N-e-(N-a-oleoyl-L-g-glutamyl)]
  misc_feature (31)..(31): N-terminal

SEQ ID NO: 19 - protein, 30 aa, Artificial Sequence; identical to SEQ ID NO: 16
  Sequence is synthesized
  misc_feature (2)..(2): Xaa is bAla
  misc_feature (29)..(29): Xaa is bAla
  misc_feature (30)..(30): N-terminal

SEQ ID NO: 20 - protein, 31 aa, Artificial Sequence
  Sequence is synthesized
  misc_feature (20)..(20): Xaa is 26Lys[N-e-(N-a-palmitoyl-L-g-glutamyl)]
  misc_feature (31)..(31): C-terminal
HAEGTFTSDVSCYLEGQAAXEFIAWLVRGRG

SEQ ID NO: 21 - protein, 31 aa, Artificial Sequence; identical to SEQ ID NO: 20
  Sequence is synthesized
  misc_feature (20)..(20): Xaa is 26Lys[N-e-(N-a-oleoyl-L-g-glutamyl)]
  misc_feature (31)..(31): C-terminal

SEQ ID NO: 22 - protein, 31 aa, Artificial Sequence; identical to SEQ ID NO: 20
  Sequence is synthesized
  misc_feature (20)..(20): Xaa is 26Lys[N-e-(N-a-palmitoyl-L-g-glutamyl)]
  misc_feature (31)..(31): N-terminal

SEQ ID NO: 23 - protein, 31 aa, Artificial Sequence; identical to SEQ ID NO: 20
  Sequence is synthesized
  misc_feature (20)..(20): Xaa is 26Lys[N-e-(N-a-oleoyl-L-g-glutamyl)]
  misc_feature (31)..(31): N-terminal

SEQ ID NO: 24 - protein, 31 aa, Artificial Sequence; identical to SEQ ID NO: 7
  Sequence is synthesized
  misc_feature (20)..(20): Xaa is 26Lys[N-e-(N-a-palmitoyl-L-g-glutamyl)]
  misc_feature (31)..(31): C-terminal

SEQ ID NO: 25 - protein, 31 aa, Artificial Sequence; identical to SEQ ID NO: 7
  Sequence is synthesized
  misc_feature (20)..(20): Xaa is 26Lys[N-e-(N-a-palmitoyl-L-g-glutamyl)]
  misc_feature (31)..(31): N-terminal

SEQ ID NO: 26 - protein, 31 aa, Artificial Sequence; identical to SEQ ID NO: 7
  Sequence is synthesized
  misc_feature (20)..(20): Xaa is 26Lys[N-e-(N-a-oleoyl-L-g-glutamyl)]
  misc_feature (31)..(31): C-terminal

SEQ ID NO: 27 - protein, 31 aa, Artificial Sequence; identical to SEQ ID NO: 7
  Sequence is synthesized
  misc_feature (20)..(20): Xaa is 26Lys[N-e-(N-a-oleoyl-L-g-glutamyl)]
  misc_feature (31)..(31): N-terminal

SEQ ID NO: 28 - protein, 31 aa, Artificial Sequence; identical to SEQ ID NO: 11
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (31)..(31): N-terminal

SEQ ID NO: 29 - protein, 31 aa, Artificial Sequence; identical to SEQ ID NO: 11
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): N-terminal

SEQ ID NO: 30 - protein, 31 aa, Artificial Sequence; identical to SEQ ID NO: 11
  Sequence is synthesized
  misc_feature (2)..(2): Xaa is bAib
  misc_feature (20)..(20): Xaa is 26Lys[N-e-(N-a-oleoyl-L-g-glutamyl)]
  misc_feature (31)..(31): N-terminal

SEQ ID NO: 31 - protein, 31 aa, Artificial Sequence; identical to SEQ ID NO: 11
  Sequence is synthesized
  misc_feature (2)..(2): Xaa is bAla
  misc_feature (20)..(20): Xaa is 26Lys[N-e-(N-a-oleoyl-L-g-glutamyl)]
  misc_feature (31)..(31): N-terminal

SEQ ID NO: 32 - protein, 31 aa, Artificial Sequence; identical to SEQ ID NO: 20
  Sequence is synthesized
  misc_feature (20)..(20): Xaa is 26Lys[N-e-(N-a-oleoyl-L-g-glutamyl)]
  misc_feature (31)..(31): C-terminal

SEQ ID NO: 33 - protein, 31 aa, Artificial Sequence; identical to SEQ ID NO: 20
  Sequence is synthesized
  misc_feature (20)..(20): Xaa is 26Lys[N-e-(N-a-oleoyl-L-g-glutamyl)]
  misc_feature (31)..(31): N-terminal

SEQ ID NO: 34 - protein, 31 aa, Artificial Sequence; identical to SEQ ID NO: 20
  Sequence is synthesized
  misc_feature (20)..(20): Xaa is 26Lys[N-e-(N-a-palmitoyl-L-g-glutamyl)]
  misc_feature (31)..(31): C-terminal

SEQ ID NO: 35 - protein, 31 aa, Artificial Sequence; identical to SEQ ID NO: 20
  Sequence is synthesized
  misc_feature (20)..(20): Xaa is 26Lys[N-e-(N-a-palmitoyl-L-g-glutamyl)]
  misc_feature (31)..(31): N-terminal

SEQ ID NO: 36 - protein, 31 aa, Artificial Sequence
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): N-terminal
HXEGTFTSDVSCYLEGQAAXEFIAWLVRGRG

SEQ ID NO: 37 - protein, 31 aa, Artificial Sequence; identical to SEQ ID NO: 36
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (31)..(31): N-terminal

SEQ ID NO: 38 - protein, 31 aa, Artificial Sequence; identical to SEQ ID NO: 36
  Sequence is synthesized
  misc_feature (2)..(2): Xaa is bAib
  misc_feature (20)..(20): Xaa is 26Lys[N-e-(N-a-oleoyl-L-g-glutamyl)]
  misc_feature (31)..(31): N-terminal

SEQ ID NO: 39 - protein, 31 aa, Artificial Sequence; identical to SEQ ID NO: 36
  Sequence is synthesized
  misc_feature (2)..(2): Xaa is bAla
  misc_feature (20)..(20): Xaa is 26Lys[N-e-(N-a-oleoyl-L-g-glutamyl)]
  misc_feature (31)..(31): N-terminal

SEQ ID NO: 40 - protein, 31 aa, Artificial Sequence
  Sequence is synthesized
  misc_feature (20)..(20): Xaa is 26Lys[N-e-(N-a-palmitoyl-L-g-glutamyl)]
  misc_feature (28)..(28): Xaa is 26Lys[N-e-(N-a-palmitoyl-L-g-glutamyl)]
  misc_feature (31)..(31): C-terminal
HAEGTFTSDVSCYLEGQAAXEFIAWLVXGRG

SEQ ID NO: 41 - protein, 31 aa, Artificial Sequence; identical to SEQ ID NO: 40
  Sequence is synthesized
  misc_feature (20)..(20): Xaa is 26Lys[N-e-(N-a-palmitoyl-L-g-glutamyl)]
  misc_feature (28)..(28): Xaa is 26Lys[N-e-(N-a-palmitoyl-L-g-glutamyl)]
  misc_feature (31)..(31): N-terminal

SEQ ID NO: 42 - protein, 31 aa, Artificial Sequence
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (28)..(28): Xaa is 26Lys[N-e-(N-a-palmitoyl-L-g-glutamyl)]
  misc_feature (31)..(31): N-terminal
HXEGTFTSDVSCYLEGQAAXEFIAWLVXGRG

SEQ ID NO: 43 - protein, 31 aa, Artificial Sequence; identical to SEQ ID NO: 42
  Sequence is synthesized
  misc_feature (2)..(2): Xaa is bAib
  misc_feature (20)..(20): Xaa is 26Lys[N-e-(N-a-palmitoyl-L-g-glutamyl)]
  misc_feature (28)..(28): Xaa is 26Lys[N-e-(N-a-palmitoyl-L-g-glutamyl)]
  misc_feature (31)..(31): N-terminal

SEQ ID NO: 44 - protein, 31 aa, Artificial Sequence; identical to SEQ ID NO: 42
  Sequence is synthesized
  misc_feature (2)..(2): Xaa is bAla
  misc_feature (20)..(20): Xaa is 26Lys[N-e-(N-a-palmitoyl-L-g-glutamyl)]
  misc_feature (28)..(28): Xaa is 26Lys[N-e-(N-a-palmitoyl-L-g-glutamyl)]
  misc_feature (31)..(31): N-terminal

SEQ ID NO: 45 - protein, 31 aa, Artificial Sequence; identical to SEQ ID NO: 36
  Sequence is synthesized
  misc_feature (2)..(2): Xaa is aAib
  misc_feature (20)..(20): Xaa 26Lys[N-e-(N-a-palmitoyl-L-g-glutamyl)]
  misc_feature (31)..(31): C-terminal

SEQ ID NO: 46 - protein, 31 aa, Artificial Sequence; identical to SEQ ID NO: 36
  Sequence is synthesized
  misc_feature (2)..(2): Xaa is aAib
  misc_feature (20)..(20): Xaa is LyO
  misc_feature (31)..(31): C-terminal

SEQ ID NO: 47 - protein, 31 aa, Artificial Sequence; identical to SEQ ID NO: 36
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): N-terminal

SEQ ID NO: 48 - protein, 31 aa, Artificial Sequence; identical to SEQ ID NO: 36
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (31)..(31): N-terminal

SEQ ID NO: 49 - protein, 31 aa, Artificial Sequence; identical to SEQ ID NO: 36
  Sequence is synthesized
  misc_feature (2)..(2): Xaa is bAib
  misc_feature (20)..(20): Xaa is 26Lys[N-e-(N-a-oleoyl-L-g-glutamyl)]
  misc_feature (31)..(31): N-terminal

SEQ ID NO: 50 - protein, 31 aa, Artificial Sequence; identical to SEQ ID NO: 36
  Sequence is synthesized
  misc_feature (2)..(2): Xaa is bAla
  misc_feature (20)..(20): Xaa is 26Lys[N-e-(N-a-oleoyl-L-g-glutamyl)]
  misc_feature (31)..(31): N-terminal

SEQ ID NO: 51 - protein, 30 aa, Artificial Sequence
  Sequence is synthesized
  misc_feature (2)..(2): Xaa is aAib
  misc_feature (29)..(29): Xaa is aAib
  misc_feature (30)..(30): N-terminal
HXEGTFTSDVSCYLEGQAAKEFIAWLVRXR

SEQ ID NO: 52 - protein, 30 aa, Artificial Sequence; identical to SEQ ID NO: 51
  Sequence is synthesized
  misc_feature (2)..(2): Xaa is aAib
  misc_feature (29)..(29): Xaa is aAib
  misc_feature (30)..(30): C-terminal

SEQ ID NO: 53 - protein, 30 aa, Artificial Sequence
  Sequence is synthesized
  misc_feature (2)..(2): Xaa is aAib
  misc_feature (29)..(29): Xaa is aAib
  misc_feature (30)..(30): N-terminal
HXEGTFTSDVSCYLEGQAAKEFIAWLVKXR

SEQ ID NO: 54 - protein, 30 aa, Artificial Sequence; identical to SEQ ID NO: 51
  Sequence is synthesized
  misc_feature (2)..(2): Xaa is bAib
  misc_feature (29)..(29): Xaa is bAib
  misc_feature (30)..(30): N-terminal

SEQ ID NO: 55 - protein, 30 aa, Artificial Sequence; identical to SEQ ID NO: 51
  Sequence is synthesized
  misc_feature (2)..(2): Xaa is bAla
  misc_feature (29)..(29): Xaa is bAla
  misc_feature (30)..(30): N-terminal

SEQ ID NO: 56 - protein, 31 aa, Artificial Sequence
  Sequence is synthesized
  misc_feature (20)..(20): Xaa is 26Lys[N-e-(N-a-palmitoyl-L-g-glutamyl)]
  misc_feature (31)..(31): C-terminal
HAEGTFTCDVSSYLEGQAAXEFIAWLVRGRG

SEQ ID NO: 57 - protein, 31 aa, Artificial Sequence; identical to SEQ ID NO: 56
  Sequence is synthesized
  misc_feature (20)..(20): Xaa is 26Lys[N-e-(N-a-palmitoyl-L-g-glutamyl)]
  misc_feature (31)..(31): N-terminal

SEQ ID NO: 58 - protein, 31 aa, Artificial Sequence; identical to SEQ ID NO: 56
  Sequence is synthesized
  misc_feature (20)..(20): Xaa is 26Lys[N-e-(N-a-oleoyl-L-g-glutamyl)]
  misc_feature (31)..(31): C-terminal

SEQ ID NO: 59 - protein, 31 aa, Artificial Sequence; identical to SEQ ID NO: 56
  Sequence is synthesized
  misc_feature (20)..(20): Xaa is 26Lys[N-e-(N-a-oleoyl-L-g-glutamyl)]
  misc_feature (31)..(31): N-terminal

SEQ ID NO: 60 - protein, 31 aa, Artificial Sequence
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (31)..(31): C-terminal
HXEGTFTCDVSSYLEGQAAXEFIAWLVRGRG

SEQ ID NO: 61 - protein, 31 aa, Artificial Sequence; identical to SEQ ID NO: 60
  Sequence is synthesized
  misc_feature (2)..(2): Xaa is aAib
  misc_feature (20)..(20): Xaa is 26Lys[N-e-(N-a-palmitoyl-L-g-glutamyl)]
  misc_feature (31)..(31): N-terminal

SEQ ID NO: 62 - protein, 31 aa, Artificial Sequence; identical to SEQ ID NO: 60
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): N-terminal

SEQ ID NO: 63 - protein, 31 aa, Artificial Sequence; identical to SEQ ID NO: 60
  Sequence is synthesized
  misc_feature (2)..(2): Xaa is aAib
  misc_feature (20)..(20): Xaa is 26Lys[N-e-(N-a-oleoyl-L-g-glutamyl)]
  misc_feature (31)..(31): C-terminal

SEQ ID NO: 64 - protein, 30 aa, Artificial Sequence
  Sequence is synthesized
  misc_feature (2)..(2): Xaa is aAib
  misc_feature (29)..(29): Xaa is aAib
  misc_feature (30)..(30): N-terminal
HXEGTFTCDVSSYLEGQAAKEFIAWLVRXR

SEQ ID NO: 65 - protein, 30 aa, Artificial Sequence
  Sequence is synthesized
  misc_feature (2)..(2): Xaa is aAib
  misc_feature (29)..(29): Xaa is aAib
  misc_feature (30)..(30): N-terminal
HXEGTFTCDVSSYLEGQAAKEFIAWLVKXR

SEQ ID NO: 66 - protein, 31 aa, Artificial Sequence; identical to SEQ ID NO: 60
  Sequence is synthesized
  misc_feature (2)..(2): Xaa is bAib
  misc_feature (20)..(20): Xaa is 26Lys[N-e-(N-a-oleoyl-L-g-glutamyl)]
  misc_feature (31)..(31): N-terminal

SEQ ID NO: 67 - protein, 30 aa, Artificial Sequence; identical to SEQ ID NO: 64
  Sequence is synthesized
  misc_feature (2)..(2): Xaa is bAib
  misc_feature (29)..(29): Xaa is bAib
  misc_feature (30)..(30): N-terminal

SEQ ID NO: 68 - protein, 31 aa, Artificial Sequence; identical to SEQ ID NO: 60
  Sequence is synthesized
  misc_feature (2)..(2): Xaa is bAla
  misc_feature (20)..(20): Xaa is 26Lys[N-e-(N-a-oleoyl-L-g-glutamyl)]
  misc_feature (31)..(31): N-terminal

SEQ ID NO: 69 - protein, 30 aa, Artificial Sequence; identical to SEQ ID NO: 64
  Sequence is synthesized
  misc_feature (2)..(2): Xaa is bAla
  misc_feature (29)..(29): Xaa is bAla
  misc_feature (30)..(30): N-terminal

SEQ ID NO: 70 - protein, 62 aa, Artificial Sequence
  Sequence is synthesized
  misc_feature (20)..(20): Xaa is 26Lys[N-e-(N-a-oleoyl-L-g-glutamyl)]
  misc_feature (62)..(62): C-terminal
HAEGTFTSDVSCYLEGQAAXEFIAWLVRGRGHGEGTFTSDVSSYLEGQAAREFIAWLVRGRG

SEQ ID NO: 71 - protein, 30 aa, Artificial Sequence
  Sequence is synthesized
  misc_feature (30)..(30): N-terminal is modified with PEG
HAEGTFTSDVSCYLEGQAAKEFIAWLVRGR